SEQUENCE LISTING (58 sequences)

SEQ ID NO: 1 - protein, 74 aa, Pseudomonas phage
MSSRDPYRIGHRVGLVNYSDRYLGADAAGTKGTIEAITRPSRCMTIYHVRCERTLRLIEAEARNVRFIRQRAER

SEQ ID NO: 2 - protein, 74 aa, Pseudomonas phage
MSSRDPYRIGHRVGLVNYSDRYLGADAAGTKGIIEAITRPSRCMTVYHVRCERTLRLIEAEARNVRFIRQRAER

SEQ ID NO: 3 - protein, 75 aa, Pseudomonas phage
MSSRDPYRIGHRVGLVNYSDRYLGADAAGTKGIIEAITRPSRCMTIYHVRCERTLRLIEAEARNVRFIRQLPTRK

SEQ ID NO: 4 - protein, 75 aa, Pseudomonas phage
MSSRDPYRIGHRVGLVNYSDRYLGADAAGTKGIIEAITRPSRCMTIYHVRCERTLRLIEAEARNVRFIRQLPTHK

SEQ ID NO: 5 - protein, 75 aa, Pseudomonas phage
MSSRDPYRIGHRVGLVNYSDRYLGADAAGTKGTIEAITRPSRCMTIYHVRCERTLRLIAAEARNVRFVRQLPTRK

SEQ ID NO: 6 - protein, 75 aa, Pseudomonas phage
MSSRDPYRIGHRVGLVNYSDRYLGADAAGTKGIIEAITRPSRCMTVYHVRCERTLRLIEAEARNVRFIRQLPTRK

SEQ ID NO: 7 - protein, 75 aa, Pseudomonas phage
MSSRDPYRIGHRVGLVNYSDRYLGADAAGTRGIIEAITRPSRCMTIYHVRCERTLRLIEAEARNVRFIRQLPTRK

SEQ ID NO: 8 - protein, 75 aa, Pseudomonas phage
MYSHDQYRIGHRVGLVNYSDRYLGADEAGTRGIVEAVTKPSRCMTVYHVRCEQTLRLIEAEARNMRFIMQPPTRK

SEQ ID NO: 9 - protein, 72 aa, Pseudomonas phage
MFLHDRFDRHLGARVGLVNYSDRYLGADSAGVKGRIEAITKPSRMMTVYHIRCEQTARLVEAEEKNMRWLGR

SEQ ID NO: 10 - protein, 123 aa, Pseudomonas phage
MKASTCCWSRAARCPATSPARRTAGSTLNTTRAARTGARSSTLQELRTMSSRDPYRIGHRVGLVNYSDRYLGADAAGTKGIIEAITRPSRCMTIYHVRCERTLRLIEAEARNVRFIRQLPTHK

SEQ ID NO: 11 - protein, 61 aa, Pseudomonas phage
RIGHRVGLVNYSDRYLGADAAGTKGTIEAITRPSRCMTIYHVRCERTLRLIEAEARNVRFI

SEQ ID NO: 12 - protein, 61 aa, Pseudomonas phage
RIGHRVGLVNYSDRYLGADAAGTKGIIEAITRPSRCMTVYHVRCERTLRLIEAEARNVRFI

SEQ ID NO: 13 - protein, 61 aa, Pseudomonas phage
RIGHRVGLVNYSDRYLGADAAGTKGIIEAITRPSRCMTIYHVRCERTLRLIEAEARNVRFI

SEQ ID NO: 14 - protein, 61 aa, Pseudomonas phage; identical to SEQ ID NO: 13

SEQ ID NO: 15 - protein, 61 aa, Pseudomonas phage
RIGHRVGLVNYSDRYLGADAAGTKGTIEAITRPSRCMTIYHVRCERTLRLIAAEARNVRFV

SEQ ID NO: 16 - protein, 61 aa, Pseudomonas phage; identical to SEQ ID NO: 12

SEQ ID NO: 17 - protein, 61 aa, Pseudomonas phage
RIGHRVGLVNYSDRYLGADAAGTRGIIEAITRPSRCMTIYHVRCERTLRLIEAEARNVRFI

SEQ ID NO: 18 - protein, 61 aa, Pseudomonas phage
RIGHRVGLVNYSDRYLGADEAGTRGIVEAVTKPSRCMTVYHVRCEQTLRLIEAEARNMRFI

SEQ ID NO: 19 - protein, 61 aa, Pseudomonas phage
HLGARVGLVNYSDRYLGADSAGVKGRIEAITKPSRMMTVYHIRCEQTARLVEAEEKNMRWL

SEQ ID NO: 20 - protein, 15 aa, artificial sequence
  conserved fragment of minDNAses
RVGLVNYSDRYLGAD

SEQ ID NO: 21 - DNA, 222 bp, Pseudomonas phage
atgtcctcgcgtgatccctaccgcatcggccaccgcgtggggctgatgaactacagcgaccgctacctgggtgccgacgcggcaggcaccaagggcaccatcgaagccataacccgaccgtcccgctgtatgacgatctaccacgtgcgctgtgagcggaccctgcgcctgatcgaggccgaggcccgcaacgtgcgattcatccgacagcgggcggagcgg

SEQ ID NO: 22 - DNA, 18 bp, artificial sequence
  primer
atgtcctcgcgtgatccc

SEQ ID NO: 23 - DNA, 16 bp, artificial sequence
  primer
ccgctccgcccgctgt

SEQ ID NO: 24 - DNA, 26 bp, artificial sequence
  primer
ataggatccatgtcctcgcgtgatcc

SEQ ID NO: 25 - DNA, 27 bp, artificial sequence
  primer
atagaattctcaccgctccgcccgctg

SEQ ID NO: 26 - DNA, 249 bp, artificial sequence
  miniDNAse + His Tag
atgtcctcgcgtgatccctaccgcatcggccaccgcgtggggctggtgaactacagcgaccgctacctgggtgccgacgcggcaggcaccaagggcaccatcgaagccataacccgaccgtcccgctgtatgacgatctaccacgtgcgctgtgagcggaccctgcgcctgatcgaggccgaggcccgcaacgtgcgattcatccgacagcgggcggagcggaagggtcatcatcaccatcaccattga

SEQ ID NO: 27 - protein, 82 aa, artificial sequence
  miniDNAse + His Tag
MSSRDPYRIGHRVGLVNYSDRYLGADAAGTKGTIEAITRPSRCMTIYHVRCERTLRLIEAEARNVRFIRQRAERKGHHHHHH

SEQ ID NO: 28 - DNA, 990 bp, artificial sequence
  GST + miniDNase fusion
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccggaattcccgggtcgactcgagcggccgcatcgtgacctggaagttctgttccaggggcccctgggatccatgtcctcgcgtgatccctaccgcatcggccaccgcgtggggctggtgaactacagcgaccgctacctgggtgccgacgcggcaggcaccaagggcaccatcgaagccataacccgaccgtcccgctgtatgacgatctaccacgtgcgctgtgagcggaccctgcgcctgatcgaggccgaggcccgcaacgtgcgattcatccgacagcgggcggagcggtga

SEQ ID NO: 29 - protein, 305 aa, artificial sequence
  GST + miniDNase fusion
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSMSSRDPYRIGHRVGLVNYSDRYLGADAAGTKGTIEAITRPSRCMTIYHVRCERTLRLIEAEARNVRFIRQRAER

SEQ ID NO: 30 - DNA, 22 bp, artificial sequence
  primer
caccatgtcctcgcgtgatccc

SEQ ID NO: 31 - DNA, 18 bp, artificial sequence
  primer
tcaccgctccgcccgctg

SEQ ID NO: 32 - DNA, 31 bp, artificial sequence
  primer
atcatgccataccgcgaaaggttttgcacca

SEQ ID NO: 33 - DNA, 20 bp, artificial sequence
  primer
ggaggggtggaaatggagtt

SEQ ID NO: 34 - DNA, 21 bp, artificial sequence
  primer
gcaccatcggagccataaccc

SEQ ID NO: 35 - DNA, 16 bp, artificial sequence
  primer
ccttggtgcctgccgc

SEQ ID NO: 36 - DNA, 19 bp, artificial sequence
  primer
cggcaggccccaagggcac

SEQ ID NO: 37 - DNA, 23 bp, artificial sequence
  primer
cgtcggcacccaggtagcggtcg

SEQ ID NO: 38 - DNA, 21 bp, artificial sequence
  primer
cataacccgaccgtcccgctg

SEQ ID NO: 39 - DNA, 26 bp, artificial sequence
  primer
gcttcgatggttctcttggtgcctgc

SEQ ID NO: 40 - DNA, 25 bp, artificial sequence
  primer
ataggatccatgtactcgcatgacc

SEQ ID NO: 41 - DNA, 25 bp, artificial sequence
  primer
atagaattctcatttacgggtcggc

SEQ ID NO: 42 - DNA, 30 bp, Artificial Sequence
  PCR primer
ataggatccatgttcttgcatgaccggttc

SEQ ID NO: 43 - DNA, 25 bp, Artificial Sequence
  PCR primer
tatgaattctcagcgccccagccag

SEQ ID NO: 44 - protein, 70 aa, Pseudomonas phage
MSSRDPYRIGHRVGLVNYSDRYLGADAAGTKGTIEAITRPSRCMTIYHVRCERTLRLIEAEARNVRFIRQ

SEQ ID NO: 45 - protein, 68 aa, Pseudomonas phage
MSSRDPYRIGHRVGLVNYSDRYLGADAAGTKGTIEAITRPSRCMTIYHVRCERTLRLIEAEARNVRFI

SEQ ID NO: 46 - protein, 64 aa, Pseudomonas phage
MSSRDPYRIGHRVGLVNYSDRYLGADAAGTKGTIEAITRPSRCMTIYHVRCERTLRLIEAEARN

SEQ ID NO: 47 - protein, 52 aa, Pseudomonas phage
MSSRDPYRIGHRVGLVNYSDRYLGADAAGTKGTIEAITRPSRCMTIYHVRCE

SEQ ID NO: 48 - protein, 68 aa, Pseudomonas phage
MRIGHRVGLVNYSDRYLGADAAGTKGTIEAITRPSRCMTIYHVRCERTLRLIEAEARNVRFIRQRAER

SEQ ID NO: 49 - protein, 64 aa, Pseudomonas phage
MRIGHRVGLVNYSDRYLGADAAGTKGTIEAITRPSRCMTIYHVRCERTLRLIEAEARNVRFIRQ

SEQ ID NO: 50 - DNA, 28 bp, Artificial Sequence
  PCR primer
tgagaattcccgggtcgactcgagcggc

SEQ ID NO: 51 - DNA, 17 bp, Artificial Sequence
  PCR primer
catggatcccaggggcc

SEQ ID NO: 52 - DNA, 21 bp, Artificial Sequence
  PCR primer
tgaaagggtgggcgcgccgac

SEQ ID NO: 53 - DNA, 31 bp, Artificial Sequence
  PCT primer
catggtgaagggctccttcttaaagttaaac

SEQ ID NO: 54 - DNA, 27 bp, Artificial Sequence
  PCR phage
ctgtcggatgaatcgcacgttgcgggc

SEQ ID NO: 55 - DNA, 25 bp, PCR primer
gatgaatcgcacgttgcgggcctcg

SEQ ID NO: 56 - DNA, 19 bp, Artificial Sequence
  PCR primer
gttgcgggcctcggcctcg

SEQ ID NO: 57 - DNA, 32 bp, Artificial Sequence
  PCR primer
ctcacagcgcacgtggtagatcgtcatacagc

SEQ ID NO: 58 - DNA, 15 bp, Artificial Sequence
  PCR primer
cgcatcggccaccgc

(19) United States
(12) Patent Application Publication
(10) Pub. No.: US 2020/0199554 A1
(43) Pub. Date: Jun. 25, 2020

(54) NOVEL DNASE

(71) Applicant: KATHOLIEKE UNIVERSITEIT LEUVEN, Leuven (BE)

(72) Inventors: Abram Aertsen, Dilsen-Stokkem (BE); Hanne Hendrix, Heverlee (BE); Rob Lavigne, Ekeren (BE); Marie Van Der Gucht, Leuven (BE)

(21) Appl. No.: 16/634,737

(22) PCT Filed: Jul. 26, 2018
(86) PCT No.: PCT/EP2018/070253

§ 371 Date: Jan. 28, 2020

(30) Foreign Application Priority Data
Jul. 28, 2017 (GB) 1712145.0

(51) Int. Cl. C12N 9/22; C07K 16/40

(52) U.S. Cl. CPC C12N 9/22; A61K 38/00; C07K 16/40

(57) ABSTRACT

The present invention relates to a group of proteins from bacterial phages which show DNA degrading activity.

### FIELD OF THE INVENTION

The present invention relates to the in vivo and in vitro use of previously uncharacterized viral proteins as DNA degrading enzymes.

### BACKGROUND OF THE INVENTION

DNases find their application in the medical field and in industrial applications. These enzymes typically work in a narrow pH range, are temperature sensitive and require specific buffer conditions. There is a need for more robust DNases. Genomic sequencing of phage genomes and analysis of Open Reading Frames (ORFs) has been performed. See for example Lavigne et al. (2003) Virology 312, 49-59; Karumidze et al. (2012) Appl Microbiol Biotechnol. 94, 1609-1617 and Ceyssens et al. (2006) J. Bacteriol. 188, 6924-6931. However, many of the characterized open reading frames have unknown functions. LUZ19_Gp5 [SEQ ID NO:1] is an uncharacterized ORF of 74 amino acids sharing sequence similarity only with other hypothetical sequences of Pseudomonas phages. In silico sequence analysis shows no significant sequence identity with other proteins and reveals no sequence motifs or protein domains which give a hint of the function of this protein.

### SUMMARY OF THE INVENTION

The invention is summarized in the following statements:

1. The in vitro use of a polypeptide comprising a sequence with at least 60% identity with any one of the sequences with SEQ ID NO:1 to SEQ ID NO:9 or comprising a fragment thereof, wherein said polypeptide or fragment thereof has nuclease activity, as a DNA degrading enzyme.

2. The in vitro use according to statement 1, wherein the polypeptide is a fusion protein with said polypeptide or fragment thereof having nuclease activity.

3. The in vitro use of a polypeptide comprising a sequence with at least 60% identity with any one of the sequences with SEQ ID NO:1 to SEQ ID NO:9 or a fragment thereof, wherein said polypeptide or fragment thereof has nuclease activity, for the degradation of biofilms.

4. An in vitro method of degrading DNA comprising the step of contacting a DNA comprising sample with a polypeptide comprising a sequence at least 60% identity with any one of the sequences with SEQ ID NO:1 to SEQ ID NO:9 or a fragment thereof, wherein said polypeptide or fragment thereof has nuclease activity.

5. The method according to statement 4, wherein the method is performed at a pH below 4, below 3 or below 2, or above 8, above 9 or above 10, e.g. between ph 1 to 4, between 2 to 4, between 3 to 4, between 8 to 10 or between 8 to 9.

6. The method according to statement 4 or 5, wherein the method is performed at a temperature above 7° C., above 80° C. or above 90° C.

7. The in vitro use of a polypeptide comprising a sequence with at least 60% identity with any one of the sequences with SEQ ID NO:1 to SEQ ID NO:9 or a fragment thereof, wherein said polypeptide or fragment thereof has nuclease activity, as an antimicrobial agent.

8. The in vitro use according to any one of statements 1 to 7, wherein the polypeptide comprises a sequence with at least 70 or 75% identity with any one of the sequences with SEQ ID NO:1 to SEQ ID NO:9 or a fragment thereof wherein said polypeptide or fragment thereof has nuclease activity.

9. The in vitro use according to any one of statements 1 to 7, wherein the polypeptide comprises a sequence with at least 80 or 85% identity with any one of the sequences with SEQ ID NO:1 to SEQ ID NO:9 or a fragment thereof wherein said polypeptide or fragment thereof has nuclease activity.

10. The in vitro use according to any one of statements 1 to 7, wherein the polypeptide comprises a sequence with at least 90, 95, 97, 98 or 99% identity with any one of the sequences with SEQ ID NO:1 to SEQ ID NO:9 or a fragment thereof wherein said polypeptide or fragment thereof has nuclease activity.

11. A polypeptide comprising a sequence with at least 60% identity with any one of the sequences with SEQ ID NO:1 to SEQ ID NO:9 or a fragment thereof, wherein said polypeptide or fragment thereof has nuclease activity for use as medicament.

12. A polypeptide comprising a sequence with at least 60% identity with any one of the sequences with SEQ ID NO:1 to SEQ ID NO:9 or a fragment thereof, wherein said polypeptide or fragment thereof has nuclease activity for use in the treatment of cystic fibrosis.

13. A polypeptide comprising a sequence with at least 60% identity with any one of the sequences with SEQ ID NO:1 to SEQ ID NO:9 or a fragment thereof, wherein said polypeptide or fragment thereof has nuclease activity for use in the treatment or prevention of bacterial infections.

14. The polypeptide for use according to any one of statements 11 to 13, wherein the polypeptide comprises a sequence with at least 70 or 75% identity with any one of the sequences with SEQ ID NO:1 to SEQ ID NO:9 or a fragment thereof wherein said polypeptide or fragment thereof has nuclease activity.

15. The polypeptide for use according to any one of statements 11 to 13, wherein the polypeptide comprises a sequence with at least 80 or 85% identity with any one of the sequences with SEQ ID NO:1 to SEQ ID NO:9 or a fragment thereof wherein said polypeptide or fragment thereof has nuclease activity.

16. The polypeptide for use according to any one of statements 11 to 13, wherein the polypeptide comprises a sequence with at least 90, 95, 98 or 99% identity with any one of the sequences with SEQ ID NO:1 to SEQ ID NO:9 or a fragment thereof wherein said polypeptide or fragment thereof has nuclease activity.

17. A polypeptide comprising a sequence with at least 60% identity with any one of the sequences with SEQ ID NO:1 to SEQ ID NO:9 or a fragment thereof, wherein said polypeptide or fragment thereof has nuclease activity, with the proviso that said polypeptide is not a polypeptide consisting of a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

18. A polypeptide according to statement 17, with the proviso that said sequence does not comprise a sequence consisting of any one of SEQ ID NO:1 to SEQ ID NO:9.

19. The polypeptide according to statement 17 or 18, with at least 70% identity, at least 80%, at least 90% identity with any one of the sequences with SEQ ID NO:1 to SEQ ID NO:9 or a fragment thereof, wherein said polypeptide or fragment thereof has nuclease activity.

20. The polypeptide according to any one of statement 17 to 19, with at least 70% identity, at least 80%, at least 90% identity with any one of the sequences with SEQ ID NO:11 to SEQ ID NO:19 or a fragment thereof, wherein said polypeptide or fragment thereof has nuclease activity.

21. The polypeptide according to any one of statements 17 to 20, which comprises the sequence RVGLVNYSDRYLGAD [SEQ ID NO:20].

22. The polypeptide according to any one of statement 17 to 21, wherein said fragment is at least 50, at least 60 or at least 70 amino acids.

23. A polynucleotide comprising a sequence encoding a polypeptide according to any one of statements 17 to 22.

24. A vector comprising a polynucleotide according to statement 24.

25. An antibody specifically binding to a polypeptide comprising a sequence with at least 60% identity with any one of the sequences with SEQ ID NO:1 to SEQ ID NO:9 or a fragment thereof, wherein said polypeptide or fragment thereof has nuclease activity.

26. The antibody according to statement 25, binding to a polypeptide comprising a sequence with at least 70 or 75% identity with any one of the sequences with SEQ ID NO:1 to SEQ ID NO:9 or a fragment thereof, wherein said polypeptide or fragment thereof has nuclease activity.

27. The antibody according to statement 25, binding to a polypeptide comprising a sequence with at least 80 or 85% identity with any one of the sequences with SEQ ID NO:1 to SEQ ID NO:9 or a fragment thereof, wherein said polypeptide or fragment thereof has nuclease activity.

28. The antibody according to statement 25, binding to a polypeptide comprising a sequence with at least 90, 95, 98 or 99% identity with any one of the sequences with SEQ ID NO:1 to SEQ ID NO:9 or a fragment thereof, wherein said polypeptide or fragment thereof has nuclease activity.

29. The antibody according to any one of statements 25 to 28, wherein the antibody specifically binds to the sequence RVGLVNYSDRYLGAD [SEQ ID NO:20].

30. A method of identifying modified versions of a polypeptide selected from the group consisting of SEQ NO: 1 to 9: with nuclease activity, comprising the steps of:

introducing one or more changes in the nucleotide sequence of a polynucleotide encoding a sequence selected from the group consisting of SEQ NO: 1 to 9, the one or more changes resulting in a modified amino acid sequence;

expressing the protein with the modified amino acid sequence;

assaying the expressed protein for nuclease activity.

31. The method according to statement 30, wherein the one or more changes are substitutions, insertion or deletions.

32. The method according to statement 30 or 31, wherein the one or more changes are a deletion at the 5′ or the 3′ end of the nucleotide sequence resulting in truncated form of a polypeptide selected from the group consisting of SEQ NO: 1 to 9.

The present invention discloses the identification of a class of viral proteins as DNA degrading enzymes. The proteins have a remarkably low Mr and are active in a broad pH range, at high temperatures and in denaturing conditions such as 200 mM guanidium chloride.

### BRIEF DESCRIPTION OF THE FIGURES

FIG. 1 shows the purification of GST- and His-tagged LUZ19_Gp5 protein.

FIG. 2 shows the purification of GST-tagged LUZ19_Gp5 protein.

FIG. 3 shows nuclease activity of LUZ19_Gp5 on various sources of DNA.

FIG. 4 shows a zymogram of LUZ19_Gp5 nuclease activity.

FIG. 5 shows the pH stability of DNase I and LUZ19_Gp5.

FIG. 6 shows temperature stability of DNase I and LUZ19_Gp5.

FIG. 7 shows activity of DNaseI and LUZ19_Gp5 under various buffer conditions.

FIG. 8 shows the effect of LUZ19_Gp5 expression in P. aeruginosa cells.

FIG. 9 shows in vivo nuclease activity of LUZ19_Gp5 in P. aeruginosa cells.

FIG. 10 shows LUZ19_Gp5 mutants with decreased toxicity on P. aeruginosa cells.

FIG. 11 shows nuclease activity of LUZ19_Gp5 mutants.

FIG. 12 show a sequence alignment of LUZ19_Gp5 and related sequences. FIG. 12A: full length sequences, 12B: N- and C-terminal truncated fragments.

FIG. 13 shows nuclease activity of phiKMV_Gp5.

FIG. 14 shows nuclease activity of LKD16_Gp5. Panel A: SDS-PAGE analysis of the purified protein. Panel B : agarose gel electrophoresis based nuclease activity test performed.

FIG. 15 shows truncation mutants of LUZ19_Gp5. A: secondary structure; B: sequence alignment.

FIG. 16 shows phenotypic effects of LUZ19_gp5 truncation mutant expression on P. aeruginosa PAO1 growth.

FIG. 17 shows phenotypic effects of the expression of LUZ19_gp5 and truncation mutants on P. putida KT2440 growth.

FIG. 18 shows quantitative assessment of the in vitro nuclease activity of LUZ19_Gp5.

### DETAILED DESCRIPTION OF THE INVENTION

### Definitions

In the context of the present invention a protein has “nuclease activity” if after 30 minutes incubation at least 10% of double stranded bacterial DNA is degraded, measured as % decrease of the intensity on an agarose gel.

In the context of the present invention “miniDNase” is a polypeptide comprising a sequence with at least 60% identity with any one of the sequences with SEQ ID NO:1 to SEQ ID NO:9 or comprising a fragment thereof, wherein said polypeptide or fragment thereof has nuclease activity.

Based on SEQ ID NO:1, eight other highly similar protein sequences have been identified in protein sequence databases and in translated DNA sequences databases. These sequences as such are known. Since their function is not known is it summited that proteins with a protein sequence depicted by any of SEQ ID NO:1 to 9 are accidental disclosures of a group of broader group of compounds with DNase activity which are proteins with similarity to SEQ ID NO: 1 to 8, or fragments thereof. Proteins with SEQ ID NO: 1 to 8, as such are accidental disclosures and are disclaimed from compound claims.

Database screening was performed up to Jul. 14, 2017 in patent and non-patent databases, and it is believed that all publicly available sequences have been retrieved. However any another sequence will be disclaimed as an undisclosed disclaimer of accidental prior art which is irrelevant for inventive step.

It is emphasized that prior art sequences are only relevant for the novelty of a compound claim on proteins or polynucleotides. Any use of this group of enzymes, including those with published protein and DNA sequences, which involves DNA degradation is novel and invention, making a disclaimer in a use or method claim unnecessary.

The sequences of SEQ ID NO:1 to 9 are translated sequences of DNA of viral genomes. The sequencing of these genomes is believed to be accurate, although sequence errors may occur. The sequence information contained in the database allows to design primers to re-sequence the genomic sequence and to correct for eventual sequences. The invention thus discloses variants of SEQ ID NO:1 to 9 and their use, wherein the variants are obtainable by sequencing Pseudomonas phage DNA using primers adjacent to or within the ORF of the miniDNase sequence. e.g. SEQ ID NO 10: is a sequence which comes into account for resequencing.

The present invention describes the characterization of proteins originally identified merely as open reading frames with unknown function from bacterial viruses which infect Pseudomonas aeruginosa. These proteins are represented by a group of sequence with SEQ ID NO:1 to 9.

These proteins have a length of between 72 to 75 amino acids.

Within the sequence databases a protein sequence can be found which has a length of 123 amino acids [SEQ ID NO: 10]. The C terminal part of this sequence is identical to the sequence of SEQ ID NO:4.

Depending on the expression system the amino terminal methionine may be removed by amino peptidase.

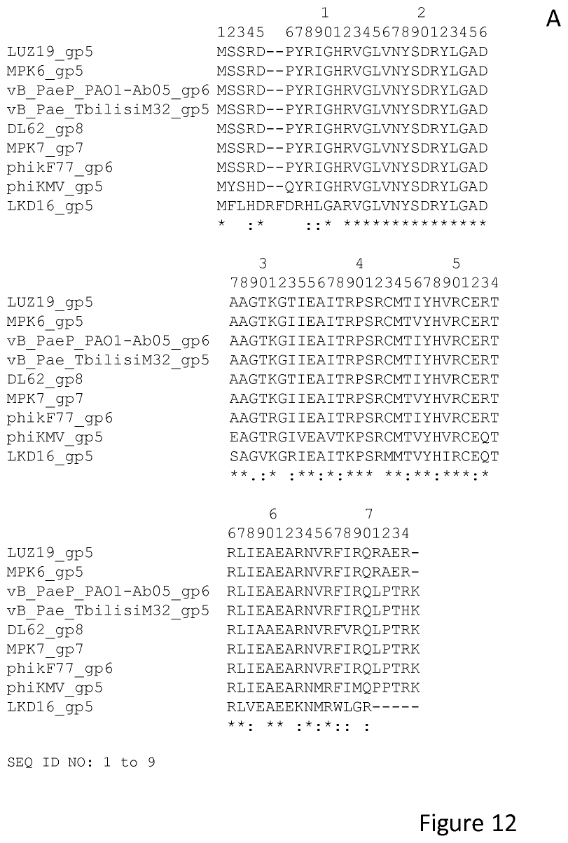

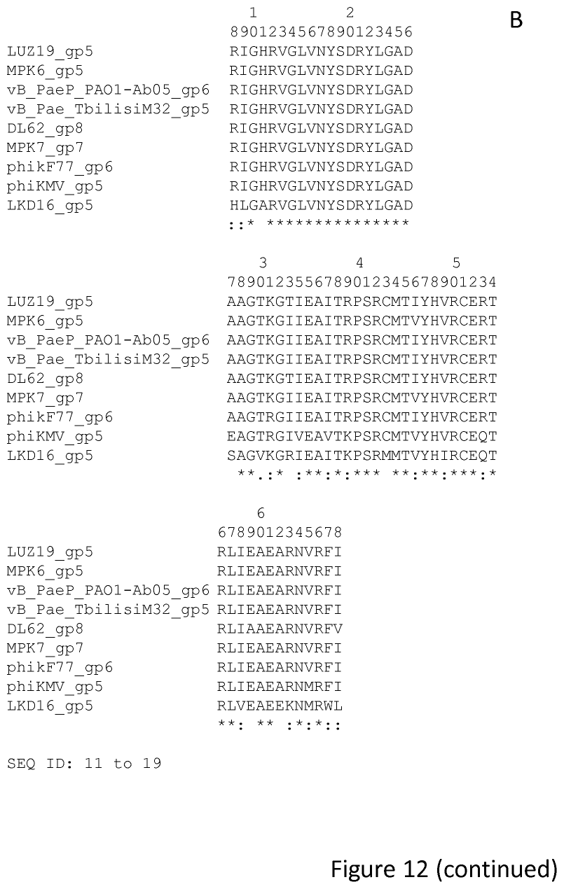

A sequence alignment (see FIG. 12) illustrates that 40 amino acids are identical in all sequences. Despite this high conservation sequence, pairwise sequence similarity may be as low as 61% for SEQ ID:NO 5 or SEQ ID NO:7 compared to SEQ ID NO:9. Indeed SEQ ID: NO 9 shows at most 65% sequence identity with any one of SEQ ID NO:1 to 8.

Within the groups of SEQ ID NO: 1 to 8, pairwise sequence identity ranges from 78 to 99%. SEQ ID NO: 8 has a pairwise sequence identity with any of SEQ ID NO: 1 to 7 of between 78 and 84%.

Despite a sequence identity of only 78% between SEQ ID NO:1 and SEQ ID NO: 8, both proteins show nuclease activity as explained in the examples.

Within the groups of SEQ ID NO: 1 to 7, pairwise sequence identity ranges from 90 to 99%.

In the group of SEQ ID NO:1 to SEQ:9, sequence variation especially occurs in the N terminal and C terminal parts. FIG. 12B shows an alignment of N and C terminal truncated versions depicted by SEQ ID NO: 11 to 19 [SEQ ID NO:11 is the truncated version of SEQ ID No:1, etc.]. Sequence identity ranges from 68 to 100% within SEQ ID NO 11: to 19. SEQ ID NO: 19 has a sequence identity of between 68 and 72 upon pairwise alignment with any of SEQ ID NO 11: to 18. Sequence identity ranges from 81 to 100% within SEQ ID NO 11: to 18. SEQ ID NO: 18 has a sequence identity of between 81% and 88% upon pairwise alignment with any of SEQ ID NO 11: to 17.

Sequence identity ranges from 93 to 100% within SEQ ID NO 11: to 17.

Within the full length sequences of SEQ ID:NO 1 to 9 and the truncated version of SEQ ID NO: 11 to 19 all sequences contain the sequence RVGLVNYSDRYLGAD [SEQ ID NO: 20].

Accordingly, the below described in vivo and in vitro uses can be performed with a protein comprising a sequence as described in any one of SEQ ID NO:1 to 9, or a sequence with at least 60, 65, 70, 75, 80, 85, 90, 95, 97% sequence identity with any of these sequences, and having nuclease activity.

The below described in vivo and in vitro uses can be performed with a protein comprising a sequence as described in any one of SEQ ID NO:1 to 8, or a sequence with at least 70, 75, 80, 85, 90, 95, 97% sequence identity with any of these sequences, and having nuclease activity.

The below described in vivo and in vitro uses can be performed with a protein comprising a sequence as described in any one of SEQ ID NO:1 to 7, or a protein comprising a sequence with at least 90, 95, 97% sequence identity with any of these sequences, and having nuclease activity.

Accordingly, the below described in vivo and in vitro uses can be performed with a protein comprising a sequence as described in any one of SEQ ID NO:11 to 19, or a sequence with at least 65, 70, 75, 80, 85, 90, 95, or 97% sequence identity with any of these sequences, and having nuclease activity.

The below described in vivo and in vitro uses can be performed with a protein comprising a sequence as described in any one of SEQ ID NO:11 to 18, or a sequence with at least 75, 80, 85, 90, 95, or 97% sequence identity with any of these sequences, and having nuclease activity.

The below described in vivo and in vitro uses can be performed with a protein comprising a sequence a described in any one of SEQ ID NO:11 to 17, or a sequence with at least 65, 70, 75, 80, 85, 90, 95, or 97% sequence identity with any of these sequences, and having nuclease activity.

In specific embodiments the below described in vivo and in vitro uses can be performed with a protein comprising a sequence a described in SEQ ID NO: 1, SEQ ID NO:11, SEQ ID NO: 8 or SEQ ID NO:18 or a sequence with at least 65, 70, 75, 80, 85, 90, 95, or 97% sequence identity with any of these sequences, and having nuclease activity.

Accordingly, the below described in vivo and in vitro uses can be performed with a protein comprising a sequence as described in any one of SEQ ID NO:11 to 19, or a sequence with at least 65, 70, 75, 80, 85, 90, 95, or 97% sequence identity with any of these sequences, and having nuclease activity.

As indicated above, the protein sequences reveal the presence of a conserved sequence RVGLVNYSDRYLGAD [SEQ ID NO: 20]. Accordingly, the below described in vivo and in vitro uses can be performed with a protein comprising SEQ ID NO: 20 and having nuclease activity.

The alignment of the proteins with SEQ ID NO:1 to 9 shows 41 amino acids positions which are conserved in all sequences, and various positions with “similar” amino acids (H/R/K; V/I/L/M, F/W/Y; D/E/Q/N; G/A/S/T/C). Preliminary mutagenesis experiments have been performed (see examples) which indicate that even mutations at conserved positions wherein size and charge of the side chain are significantly changed (Gly to Arg and Glu to Gly), retain nuclease activity. Accordingly the present invention provides methods to identify whether or not a sequence with a given percentage to any one of SEQ ID NO: 1 to 9 is a protein having nuclease activity.

Specific embodiments of mutation are mutations which prevent degradation by sequence specific proteases, and mutations in the potential Asn17XaaSer19 glycosylation site upon expression in eukaryotic systems. Mutations of one or both of Cys43 and Cys51 in SEQ ID NO:1 to 8 may reveal the effect on activity, and inter and intramolecular disulfide formation. Equally Cys51 of SEQ ID NO:9 may be mutated and/or Met 43 may be modified into Cysteine.

The present invention further provides methods to identify whether truncated forms of any one of SEQ ID NO:1 to 9 retain nuclease activity. Herein, truncated forms of any one of SEQ ID NO:1 to 9 can be cloned and expressed in an expression system and the expressed truncated protein is tested for nuclease activity.

Thus the present invention envisages truncated version at the amino terminus and/or carboxyterminus of any of SEQ ID NO:1 to 9, with a length of at least 40, at least 50, at least 55, at least 60, at least 65 amino acids.

Specific truncations are fragments comprising the conserved motif with SEQ ID NO:20 and comprising the conserved cysteine at position 51, such as miniDNase fragments corresponding to amino acid 1 up to and including 51, miniDNase fragments corresponding to amino acid 8 up to and including 51, or miniDNase fragments corresponding to amino acid 12 up to and including 51.

Other fragments are C term truncations up to but excluding truncation of V65, truncations up to but excluding truncation of R66, truncations up to but excluding truncation of F67, or truncations up to but excluding truncation of I68, Other fragments are N term truncations up to but excluding truncation of R8. Other fragments are truncations at the N terminus up to but excluding truncation of R8 end at the C terminus as defined above.

Apart from proteins comprising fragments of SEQ ID NO:1 to 9 and modified versions thereof, proteins as used in the present invention may contains further polypeptide sequence such as signal peptides for secretion of the peptide, tags for antibody binding, such as HA-tags, protein sequences for affinity purification such as His Tags, MPB, GST and the like, polypeptides encoding fluorescent enzymes, or polypeptides with another enzymatic function or polypeptides with a pharmaceutical activity.

The function of the protein sequences of SEQ ID NO:1 to 9 have not been studied in the prior art. The present invention identified these sequences as nucleases. This allows to engineer modified versions of these sequences by mutagenesis and/or truncation of one of the sequences with SEQ ID NO:1 to 9, and to assay these modified versions for their DNase activity.

In the present invention, proteins with DNase activity comprising a sequence with at least 60% sequence identity with SEQ ID NO:1 to 9, or comprising fragments thereof with DNase activity, are described as miniDNase.

Aspect of the present invention relates to in vitro uses of the proteins of the present invention.

MiniDNase can be obtained in high amounts via recombinant expression in bacteria. Although this enzyme would be assumed to be toxic to the expression host, it is believed without being bound by theory that miniDNase becomes active only in the periplasm of the bacteria when disulfide bridges are formed. Expression systems wherein a signal peptide is used for transport to the periplasm and subsequent secretion are envisaged. Alternatively host strains with cytoplasmatic disulfide bridge formation are envisaged. A tightly controlled promoter and a subsequent strong induction may be needed if the miniDNase has strong activity within the bacterial host.

The fact that the miniDNase can be expressed with aminoterminal or carboxyterminal tags allows purification at a high purity. Furthermore in view of its thermal stability, eventual contaminating proteins can be heat-inactivated. The use of eukaryotic expression system such as yeast, insect cells or mammalian cells is equally envisaged.

In embodiments of the present invention the miniDNases are used in molecular biology techniques as an alternative to commercially available enzymes such as DNase I. MiniDNase is thus provides in a kit with suitable reaction buffers and optionally a component such as citrate to stop nuclease activity.

In other embodiments, miniDNase is used in in vitro applications wherein DNA degradation is needed. An example hereof is the reduction of viscosity in cell lysates of bacterial, yeast, or eukaryotic cell preparations.

Indeed, while some fermentation products are secreted by the cells and therefore automatically present in the extracellular medium, others remain intracellular, requiring cell lysis to access the desired products. However, cell lysis not only releases the fermentation products, but also other intracellular components, which can interfere with the downstream processing. In this regard, the release of the chromosomal DNA is responsible for an increase of the lysate viscosity that may reach 75% and thereby severely complicates further recovery steps.

Since the released chromosomal DNA is responsible for most of the lysate viscosity increase, it is the principal target for viscosity reduction to improve the downstream processing efficiency and production costs. In fermentation industry, this is currently achieved by an external addition of nucleases, which are relatively large nucleic acid-degrading enzymes, like Benzonase® [see table 11] (Benedik & Strych (1998) FEMS Microbiol. Lett. 165: 1-18). Furthermore, this degradation step also prevents the undesired spread of genetic material into the environment. Unfortunately, the addition of foreign biological substances, like enzymes, in the production stream is subject to stringent regulations, making this a very costly practice. Current research focusses on the production of fermentation strains that secrete nucleic acid degrading enzymes themselves to prevent an external addition step [Cooke et al. (2003) J. Biotech. 101: 229-239].

The present invention provides an alternative solution for reducing the viscosity of cell lysates, by providing a DNase that is active in wide pH and temperature range, The miniDNase can be coated on the walls of a recipient, can be immobilized on a bead, or can be added as soluble protein.

Another aspect of the present invention relates to the degradation of biofilms using a miniDNase of the present invention. These biofilms may contain, apart from extracellular matrix, extracellular DNA (eDNA).

99% of the world's bacterial population is estimated to reside in biofilms. These are surface-associated bacterial communities embedded within a matrix of self-produced extracellular polymeric substances (EPS), like polysaccharides, proteins, lipids and eDNA. This three-dimensional matrix provides unique properties to the biofilm, including efficient resource trapping and pour penetration of the biofilm by antibacterials. In addition, biofilms are characterized by the efficient spread of resistance genes, the presence of persisters and a decreased growth rate, additionally contributing to an increased resistance to antibacterials compared to their free-living counterparts. Besides occupying natural environments, biofilms are often found in an industrial and medical context, where they are usually unwanted.

Biofilms are common threats in many industrial branches. The presence of biofilms involves operational problems in many different industrial branches. For example, they are ubiquitous at the inner surface of heat exchanging tubes in cooling water systems, being able to reduce the heat change capacity up to 90%. Furthermore, surface properties in pipelines are often altered due to the adhesion of biofilms, causing not only clogging, but also an increased fluid frictional resistance, which results in substantial energy losses. In addition, biofilms can actively accelerate the corrosion of metal surfaces up to 10,000 times, referred to as biocorrosion. Finally, the presence of biofilms involves additional issues in industries directly related to animal and human health, like the drinking water and food industry. Free-living bacteria and their spores are continuously released from plant-associated biofilms into the product streams, leading to food spoilage and contamination with pathogens.

Since eDNA is ubiquitous in the biofilm matrix and crucial to the structural integrity, its degradation with nucleases is an attractive strategy for biofilm control [Okshevski et al. (2015) Current Opinion Biotechnology 33, 73-80]. On the other hand, proactive strategies focus on the prevention biofilm formation. This can be achieved by interfering with cell signalling systems that are responsible for biofilm formation, or applying coatings on medical or industrial surfaces with bactericidal, matrix-degrading or attachment-preventing properties. For example, the coating of a surface with the nuclease DNase I has been shown to reduce the adhesion of the bacteria Pseudomonas aeruginosa and Staphylococcus aureus with 99% and 95% respectively [Swartjes et al. (2013) Advanced Functional Materials. 23, 2843-2849]. Finally, also bacteriophages are currently examined as an innovative strategy for biofilm eradication. They are viruses that specifically infect bacteria, making them natural enemies. Besides being excellent killers of bacteria, some bacteriophages also efficiently penetrate the biofilm matrix by the use of matrix-degrading enzymes [Harper et al. (2014) Antibiotics 3, 270-284]. For example, Salmonella biofilms have been shown to be prevented and reduced for 90% and 66% respectively upon bacteriophage treatment [Gong & Jiang (2017) Poultry Sci. 96, 1838-1848].

The present invention thus relates to the use of a miniDNase, alone or in combination with other microbial agents to combat biofilms of bacteria or yeast. Biofilm formation can be prevented by immobilizing DNase on surfaces prone to biofilm growth. Biofilms also grow e.g. on cloths, accordingly the use of MiniDNase in washing powders and detergents is equally envisaged.

The invention thus also relates to the use of miniDNase as an antimicrobial agent. The miniDNase can be used as an antimicrobial agent in non-medical applications whereby the DNase is formulated as a soluble protein or encapsulated in liposomes. Such liposomes are e.g. described in Jones (2005) Methods Enzymol. 391, 211-228.

In certain embodiments the miniDNase is cloned in the genome of a bacteriophage such that the miniDNase protein is presented at the surface of the phage. Suitable cloning methods are known from phage display technology.

Another embodiment is the medical use of a miniDNase of the present invention. Due to their high resistance to antibacterials, as well as to the host immune system, biofilms are a major concern to animal and human health. Since 80% of the human infections are estimated to result from biofilms, there is a high demand for the development of effective control strategies. Two types of infection-causing biofilms can be distinguished. On the one hand, biofilms can attach to the surface of medical devices, like catheters, prostheses, pacemakers and contact lenses. When these devices are brought into contact with the human body, the biofilms gain access to human tissues and cause infection. On the other hand, many pathogens can cause native biofilm-associated infections in body parts that are normally germ-free.

Human DNaseI (hDNAseI) is used since decades for improving the respiratory function of cystic fibrosis patients, commercialized as Pulmozyme® [see table 11]. Despite its success, still a significant group of patients are non-responding to a hDNAse treatment or developed antibodies to the hDNAse.

Thus the present invention envisages miniDNase for the use in the alleviation of the symptoms of cystic fibrosis as monotherapy or in combination with hDNAse. Specific patients groups are non-responders to hDNase or patients who have developed an antibody response against hDNAse.

Other disorders which can be treated with the miniDNases of the present invention include lung infections outside the context of CF, bone infections and persistent wound infections (Burmolle M et al. (2010) FEMS Immunol. Medical Microbiol. 59, 324-336).

Due to its robust properties it is believed that miniDNase has advantageous properties as well in the production of aerosols as in the pharmaceutical activity of the compound. In addition, due to its low molecular weight, the protein is likely not immunogenic.

The use of phages to treat bacterial infection is an emerging field, and is applicable to e.g. skin infections, infections of the respiratory systems (such as bacterial pneumonia), but is also for the prevention and treatment of bacterial infections caused by contamination of catheters or other surgical materials such as implants. A review can be found in Barbu (2016) Cold Spring Harb Perspect Biol 8(10). An aspect of the invention is the use of a miniDNase in such phage therapy. This can be done by cloning the miniDNase as a fusion protein with a structural protein of the phage such that the phage already by entry acts on the DNA of the infected cells. Alternatively the miniDNase is cloned as a late gene in the viral genome and is released as a soluble protein.

Other embodiments of antibacterial therapy are combination therapies with miniDNase and antibacterial proteins such as the cell membrane degrading endolyins or artilysins (Briers et al. (2014) mBio. 5, e01379-14).

Other embodiments of antibacterial therapy are combination therapies with miniDNase and antibacterial peptides or antibiotics.

The invention is illustrated in the following examples.

### Example 1: LUZ19_Gp5 and Its C-Terminal His-Tagged and N-Terminal GST-Tagged Variants. Cloning, Expression and Affinity-Based Purification of the His-Tagged and GST-Tagged Gp5 of the Pseudomonas aeruginosa Phage LUZ19. Removal of the GST-Tag and Purification by Size-Exclusion

LUZ19_Gp5 (74 amino acids long, MW=8451 Da) is a hypothetical protein originating from Pseudomonas aeruginosa phage LUZ19 (Leuven UZ phage isolate 19).

The open reading frame [SEQ ID NO:21 ] and the encoded amino acid sequence [SEQ ID NO:1 ] is shown below:

```
  1 5 10
atg tcc tcg cgt gat ccc tac cgc atc ggc cac cgc
  M S S R D P Y R I G H R
                  15 20
gtg ggg ctg atg aac tac agc gac cgc tac ctg ggt
  V G L V N Y S D R Y L G
  25 30 35
gcc gac gcg gca ggc acc aag ggc acc atc gaa gcc
  A D A A G T K G T I E A
                          40 45
ata acc cga ccg tcc cgc tgt atg acg atc tac cac
  I T R P S R C M T I Y H
          50 55 60
gtg cgc tgt gag cgg acc ctg cgc ctg atc gag gcc
  V R C E R T L R L I E A
                                  65 70
gag gcc cgc aac gtg cga ttc atc cga cag cgg gcg
  E A R N V R F I R Q R A
          74
gag cgg
  E R
```

Purified genomic DNA of phage LUZ19 was used as a template for the amplification of the open reading frame (ORF5) encoding the hypothetical protein LUZ19_Gp5 in standard PCR reactions with Pfu polymerase (Thermo Scientific, Waltham, Mass., USA) or DreamTaq polymerase (Thermo Scientific). The following parameters were used:

| C-terminal His-tag | N-terminal GST-tag |
|---|---|
| (DreamTaq polymerase) | (Pfu polymerase) |
| 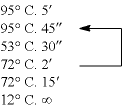 | 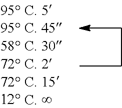 |

Forward (Primer 1) and reverse (Primer 2) primers for these PCRs are shown in Table 1.

TABLE 1
Primers used during standard PCR
amplification of LUZ19_gp5.

| | | |
|---|---|---|
| Forward | Primer 1 | ATGTCCTCGCGTGATCCC |
| primer | | [SEQ ID NO: 22] |
| Reverse | Primer 2 | CCGCTCCGCCCGCTGT |
| primers | | [SEQ ID NO: 23] |
| Forward | Primer 1 | ATAGGATCCATGTCCTCGCGTGATCC |
| primer + | | [SEQ ID NO: 24] |
| BamHI site | | |
| Reverse | Primer 2 | ATAGAATTCTCACCGCTCCGCCCGCTG |
| primer + | | [SEQ ID NO: 25] |
| EcoRI | | |

Both obtained PCR fragments were purified using the GeneJet™ PCR purification kit (Thermo Scientific) according to the manufacturer's protocol. The purified fragment of the amplified ORF for C-terminal His-tag fusion was then ligated in the commercial available pEXP5-CT/TOPO® expression vector (Invitrogen, Carlsbad, Calif., USA) following the TA-cloning protocol provided by the manufacturer, causing a fusion on the 3′/C-terminal side of the phage protein to the 6×Histidine tag (His-tag) necessary for purification. The purified fragment for N-terminal GST-tag fusion was directionally cloned in the commercial available pGEX-6P-1 vector (GE Healthcare, Little Chalfont, UK) using restriction enzymes BamHI (Thermo Scientifc) and EcoRI (Thermo Scientific), causing a fusion on the 5′/N-terminal side of the phage protein to the PreScission cleavage site and the glutathione S-transferase tag (GST-tag). After restriction of the PCR fragment and the vector in the condition suggested by the manufacturer, the linearized vector was dephosphorylated by adding 5 μl FastAP (thermosensitive alkaline phosphatase, Thermo Scientific) and incubation for 10 min at 37° C. Following inactivation (10 min, 75° C.) and purification, the DNA fragment was ligated using T4 DNA ligase (Thermo Scientific) according to the manufacturer's protocol. A 10-fold access of the insert DNA compared to the linearized vector was used. The obtained DNA and amino acid sequences for the recombinant LUZ19_Gp5 protein are shown in Table 2.

TABLE 2
Amino add and nucleotide sequences of recombinant LUZ19_Gp5 proteins

| | |
|---|---|
| Amino acid | MSSRDPYRIGHRVGLVNYSDRYLGADAAGTKGTIEAITRPSRCMTIYHVRCER |
| sequence | TLRLIEAEARNVRFIRQRAERKGHHHHHH |
| of His-tagged | [his tag underlined] [SEQ ID NO: 27] |
| LUZ19_Gp5 | |
| Nucleotide | ATGTCCTCGCGTGATCCCTACCGCATCGGCCACCGCGTGGGGCTGGTGAACTA |
| sequence | CAGCGACCGCTACCTGGGTGCCGACGCGGCAGGCACCAAGGGCACCATCGAAG |
| of His-tagged | CCATAACCCGACCGTCCCGCTGTATGACGATCTACCACGTGCGCTGTGAGCGG |
| LUZ19_Gp5 | ACCCTGCGCCTGATCGAGGCCGAGGCCCGCAACGTGCGATTCATCCGACAGCG |
| | GGCGGAGCGGAAGGGTCATCATCACCATCACCATTGA [SEQ ID NO: 26] |
| Amino acid | MSPILGYWKIKGLVQPTRLLLEYLEEKYEEHLYERDEGDKWRNKKFELGLEFP |
| sequence | NLPYYIDGDVKLTQSMAIIRYIADKHNMLGGCPKERAEISMLEGAVLDIRYGV |
| of GST-tagged | SRIAYSKDFETLKVDFLSKLPEMLKMFEDRLCHKTYLNGDHVTHPDFMLYDAL |
| LUZ19_Gp5 | DVVLYMDPMCLDAFPKLVCFKKRIEAIPQIDKYLKSSKYIAWPLQGWQATFGG |
| | GDHPPKSDLEVLFQGPLGSMSSRDPYRIGHRVGLVNYSDRYLGADAAGTKGTI |
| | EAITRPSRCMTIYHVRCERTLRLIEAEARNVRFIRQRAER |
| | [GST underlined] [SEQ ID NO: 29] |
| Nucleotide | ATGTCCCCTATACTAGGTTATTGGAAAATTAAGGGCCTTGTGCAACCCACTCG |
| sequence | ACTTCTTTTGGAATATCTTGAAGAAAAATATGAAGAGCATTTGTATGAGCGCG |
| of GST-tagged | ATGAAGGTGATAAATGGCGAAACAAAAAGTTTGAATTGGGTTTGGAGTTTCCC |
| LUZ19_Gp5 | AATCTTCCTTATTATATTGATGGTGATGTTAAATTAACACAGTCTATGGCCAT |
| | CATACGTTATATAGCTGACAAGCACAACATGTTGGGTGGTTGTCCAAAAGAGC |
| | GTGCAGAGATTTCAATGCTTGAAGGAGCGGTTTTGGATATTAGATACGGTGTT |
| | TCGAGAATTGCATATAGTAAAGACTTTGAAACTCTCAAAGTTGATTTTCTTAG |
| | CAAGCTACCTGAAATGCTGAAAATGTTCGAAGATCGTTTATGTCATAAAACAT |
| | ATTTAAATGGTGATCATGTAACCCATCCTGACTTCATGTTGTATGACGCTCTT |
| | GATGTTGTTTTATACATGGACCCAATGTGCCTGGATGCGTTCCCAAAATTAGT |
| | TTGTTTTAAAAAACGTATTGAAGCTATCCCACAAATTGATAAGTACTTGAAAT |
| | CCAGCAAGTATATAGCATGGCCTTTGCAGGGCTGGCAAGCCACGTTTGGTGGT |
| | GGCGACCATCCTCCAAAATCGGATCTGGAAGTTCTGTTCCAGGGGCCCCTGGG |
| | ATCCATGTCCTCGCGTGATCCCTACCGCATCGGCCACCGCGTGGGGCTGGTGA |
| | ACTACAGCGACCGCTACCTGGGTGCCGACGCGGCAGGCACCAAGGGCACCATC |
| | GAAGCCATAACCCGACCGTCCCGCTGTATGACGATCTACCACGTGCGCTGTGA |
| | GCGGACCCTGCGCCTGATCGAGGCCGAGGCCCGCAACGTGCGATTCATCCGAC |
| | AGCGGGCGGAGCGGTGA [SEQ ID NO: 28] |

Recombinant expression of LUZ19_Gp5 was performed in exponentially growing E. coli BL21 (DE3) cells after induction with 1 mM IPTG (isopropylthiogalactoside) at 16° C. (GST-tagged Gp5) or 30° C. (His-tagged Gp5) overnight. The phage protein was then purified using a 5 ml GSTrap HP column (GE Healthcare) or 1 ml HisTrap HP column (GE Healthcare), depending on the fused tag, on an Äkta Fast Protein Liquid Chromatograph (FPLC, GE Healthcare). The affinity chromatography is performed in four subsequent steps, all at room temperature:

For the C-terminal His-tagged protein (1 ml HisTrap HP column)

1. Equilibration of the column with 10 column volumes of Lysis Buffer (20 mM NaH2PO4, 500 mM NaCl on pH7.4) at a flow rate of 1 ml/min.

2. Loading of the total lysate (with the desired phage protein) on the HisTrap HP column at a flow rate of 0.5 ml/min.

3. Washing of the column with 10 column volumes of Washing buffer (50 mM imidazole, 20 mM NaH2PO4, 500 mM NaCl on pH7.4) at a flow rate of 1 ml/min.

4. Elution of bounded Gp5 from the column with 10 column volumes of Elution Buffer (500 mM imidazole, 20 mM NaH2PO4, 500 mM NaCl on pH7.4) at a flow rate of 0.5 ml/min.

For the N-terminal GST-tagged protein (5 ml GSTrap HP column)

1. Equilibration of the column with 2 column volumes of PBS Buffer (10 mM Na2HPO4, 1.8 mM KH2PO4, 140 mM NaCl, 10 mM KCl on pH 7.3) at a flow rate of 2 ml/min.

2. Loading of the total lysate (with the desired phage protein) on the GSTrap HP column at a flow rate of 1 ml/min.

3. Washing of the column with 5 column volumes of PBS Buffer at a flow rate of 2 ml/min.

4. Elution of bounded Gp5 from the column with 2 column volumes of Elution Buffer (50 mM Tris, 10 mM glutathione reduced on pH 8) at a flow rate of 1 ml/min.

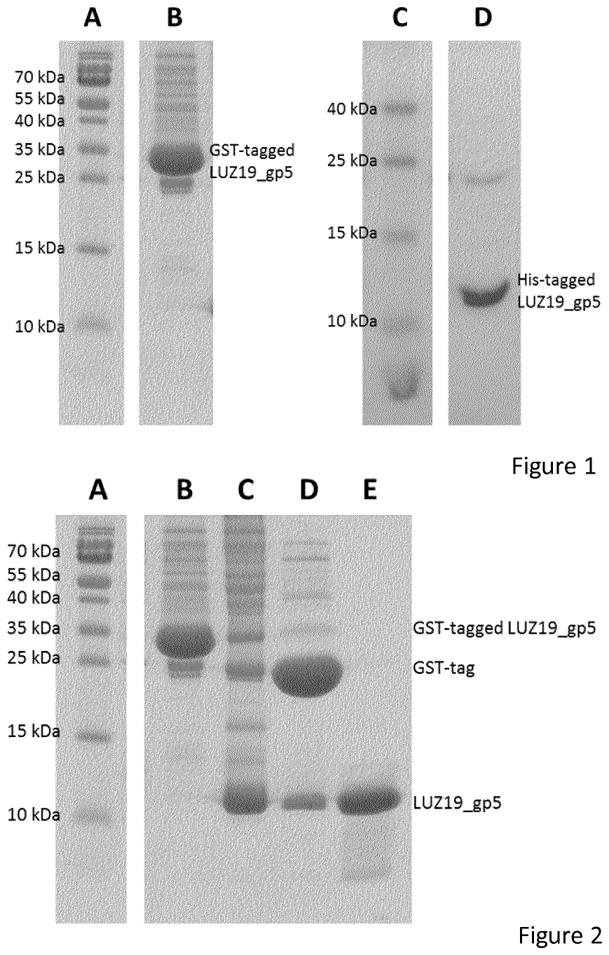

The yields for the purifications of recombinant LUZ19_Gp5 are shown in Table 3. The protein concentration was determined spectrophotometrically at a wavelength of 280 nm. Purified stock solutions of both proteins were analyzed visually on an 16% SDS-PAGE gel (FIG. 1).

TABLE 3
Yields of purified recombinant LUZ19_Gp5 protein
per liter E. coli expression culture as determined
by spectrophotometric measurement at 280 nm.

| LUZ19_Gp5 | Expression yield | |
|---|---|---|
| C-terminal His-tag | 0.4 | mg |
| N-terminal GST-tag | 28 | mg |
| Untagged, after cleavage | 0.8 | mg |

FIG. 1 shows an SDS-PAGE analysis of the purified LUZ19_Gp5 protein fused to the GST-tag (lane B) and purified LUZ19_Gp5 protein fused to the His-tag (lane D) alongside a PageRuler™ prestained protein ladder (lane A and C).

To remove the glutathione S-transferase tag, the elution fractions containing the protein of interest were first concentrated and dialysed to a Digestion Buffer (50 mM Tris, 100 mM NaCl, 1 mM ethylenediaminetetraacetic acid (EDTA), 1 mM dithiothreitol on pH 8) using Microsep™ Advance Centrifugal Device 3K (Pall Corporation, Port Washington, N.Y., USA) and Slide-A-Lyzer® MINI Dialysis Devices 3.5K 2 ml (Thermo Scientific) respectively, according to the manufacturer's protocols. Next, 1 U of the human rhinovirus 3C (HRV3C) Protease (TURBO3C, 2 U/μl, MoBiTec GmbH, Göttingen, Germany) was added per 400 μg of GST-tagged protein and incubated overnight at 4° C. to cleave the protein from the GST-tag. The final purification was done with size-exclusion chromatography using HiLoad 16/600 Superdex 75 Prep grade gel filtration column (GE Healthcare), which was calibrated using the Gel Filtration Calibration Kit LMW (GE Healthcare). After an equilibration with one column volume (124 ml) of Gel Filtration Buffer (50 mM Tris, 100 mM NaCl on pH 8) at 1 ml/min, the protein sample was loaded at the same speed and 120 1 ml fractions were collected. The samples containing the recombinant protein were finally concentrated with Amicon Ultra 3K 0.5 ml (Millipore, Ontario, Canada) and analyzed visually on an SDS-PAGE gel (FIG. 2). The final yield for the purification of the untagged Gp5 was 0.78 mg per liter E. coli expression culture (Table 3).

FIG. 2 shows an SDS-PAGE analysis of the LUZ19_Gp5 fused to the GST-tag after affinity-based purification (lane B), cleavage of the purified LUZ19_Gp5 from the GST-tag (precipitate in lane C and supernatant in lane D), and pure LUZ19_Gp5 after size-exclusion chromatography (lane E) alongside a PageRuler™ prestained protein ladder (lane A).

### Example 2: Determination and Characterization of Biochemical Nuclease Activity of P. aeruginosa Phage Protein LUZ19_Gp5 and Its N-Terminal GST-Tagged Variant

The nuclease activity was qualitatively demonstrated by using agarose gel electrophoresis and ethidium bromide staining to detect the loss of nucleic acids after exposure to the phage protein. The gels contained 1% agarose and the running buffer was TAE (40 mM Tris, 0.5 mM sodium acetate, 50 mM EDTA on pH 7.2).

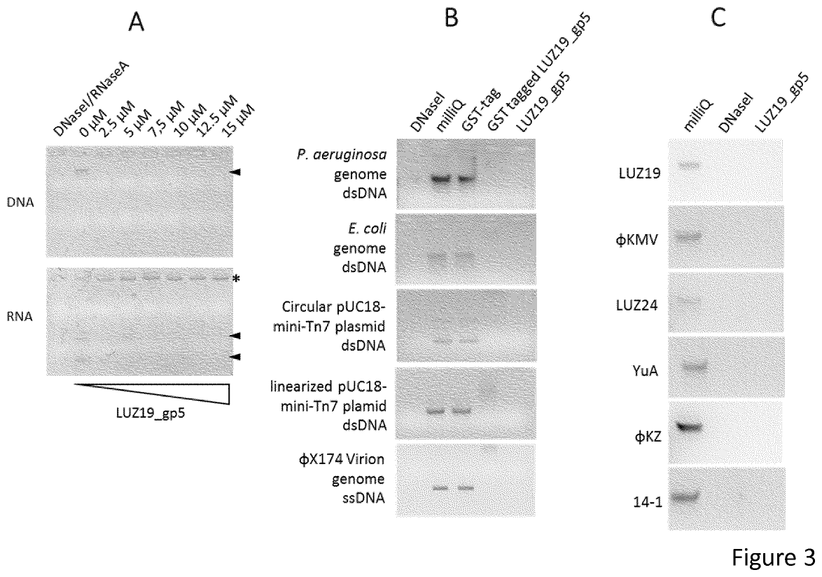

First, the degradation capacity of LUZ19_Gp5 for deoxyribonucleic acids (DNA) and ribonucleic acids (RNA) was studied. Increasing concentrations of the untagged phage protein (0-15 μM) were incubated with 100 ng host DNA (P. aeruginosa genome) or host RNA (isolated P. aeruginosa mRNA, rRNA and tRNA) in DNase I buffer+MgCl₂ (Thermo Scientific) for 30 min at 37° C. As positive controls, 1 U of the commercial DNase I (Thermo scientific) and 10 μM of the commercial RNase A (Thermo Scientific) were used. The results showed that DNA was completely degraded, even at the lowest concentration tested (2.5 μM). In addition, the phage-encoded nuclease interacted with the RNA, but no digestion was observed (for details see FIG. 3A). Indeed, the RNA remains in the loading wells which indicates that a complex formation with the protein takes place. Experiments are performed to test the degradation of single stranded RNA, double stranded RNA and DNA/RNA hybrids. Dependent on the outcome of such experiments the present invention equally envisages the in vitro use of miniDNase in the degradation of ssRNA, dsRNA or DNA/RNA hybrids.

To evaluate its specificity for various DNA molecules, the purified LUZ19_Gp5 protein was incubated with diverse deoxyribonucleic acids including the double-stranded (ds) P. aeruginosa genome, the ds E. coli genome, the ds linearized and circular pUC18-mini-Tn7-Lac-GW plasmid and the single-stranded (ss) φX174 virion genome. For this, 100 ng of nucleic acids were respectively incubated with 10 μM of the purified LUZ19_Gp5 protein in 20 μl of reaction mixture including DNase I buffer+MgCl₂ at 37° C. for 1 h. Moreover, the influence of a fusion protein on its activity was tested by using the N-terminal GST-tagged variant. The commercial DNase I was also included as a positive control and the purified GST protein as a negative control. Since all the deoxyribonucleic acids could be degraded by LUZ19_Gp5, the nuclease was described as non-specific. In addition, the nuclease was still active in presence of a N-terminal fusion protein, even when this protein was three times its size (for details see FIG. 3B).

Finally, the specificity of LUZ19_Gp5 for various bacteriophage genomes was tested. All the tested phages are Caudovirales, but belong to different families: LUZ19, φKMV, LUZ24 (Podoviridae), YuA (Siphoviridae), φKZ and 14-1 (Myoviridae). 100 ng of purified phage genomes were incubated with 10 μM of the purified LUZ19_Gp5 protein in 20 μl of reaction mixture including DNase I buffer+MgCl₂ at 37° C. for 1 h. Pure water (milliQ) and the commercial DNase I were used as a negative and a positive control respectively. The results showed that all the phage genomes were completely degraded, even the modified YuA genome and the LUZ19 genome, which contains the LUZ19_gp5 gene (for details see FIG. 3C).

FIG. 3 shows agarose gel electrophoresis based nuclease activity assays of tagged and untagged LUZ19_Gp5 with different substrates. (A) Degradation of deoxyribonucleic acids (DNA) and binding to ribonucleic acids (RNA) by increasing concentrations of untagged LUZ19_Gp5. The protein was incubated with 100 ng of nucleic acids in 20 μl reaction buffer for 30 min at 37° C. The commercial DNase I (for DNA) and RNaseA (for RNA) were included as positive controls. The protein concentrations are shown on the top and the type of nucleic acids are indicated on the left. The arrows point out the position of DNA and RNA, and a shift in migration is indicated with an asterisk. (B) Digestion of various deoxyribonucleic acids by the purified LUZ19_Gp5 and its N-terminal GST-tagged variant. MilliQ water and the purified GST protein were used as negative controls. The commercial DNase I was included as a positive control. 100 ng of nucleic acids were respectively incubated with 10 μM of the purified LUZ19_Gp5 protein in 20 μl of reaction buffer at 37° C. for 1 h. The protein solutions are shown on the top and the origin of nucleic acids are indicated on the left. (C) Degradation of various bacteriophage genomes by the purified LUZ19_Gp5. The phages are all Caudovirales, but belong to different families: LUZ19, φKMV, LUZ24 (Podoviridae), YuA (Siphoviridae), φKZ and 14-1 (Myoviridae). 100 ng of the purified phage DNA was incubated with 10 μM of the purified LUZ19_Gp5 protein in 20 μl of reaction buffer at 37° C. for 1 h.

MilliQ and the commercial DNase I were used as negative and positive control respectively.

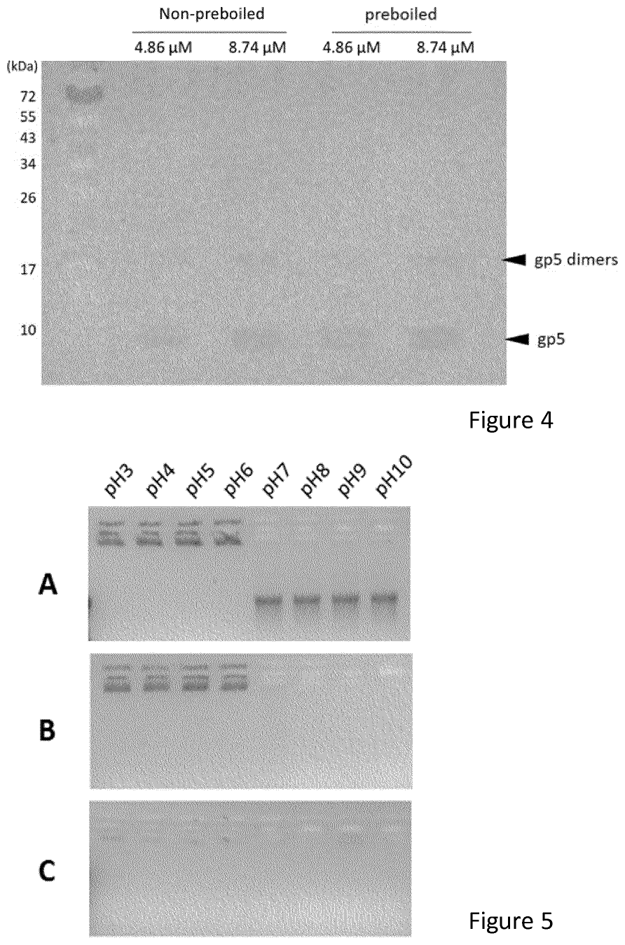

In addition, the nuclease activity was confirmed by zymography. First, samples of purified untagged LUZ19_Gp5 (4.86 and 8.74 μM) diluted in 4× SDS-PAGE loading dye (200 mM Tris HCl pH 6.8, 8 mM EDTA, 40% (v/v) glycerol, 4% (w/v) SDS, 0.4% (w/v) bromophenol blue) were separated on an 16% SDS-PAGE gel containing 100 μg/ml DNA from fish sperm (Sigma Aldrich, St. Louis, Mo., USA). Then, the gel was washed three times for 5 min in mQ and incubated for 48 h in zymogram renaturation buffer (150 mM NaH₂PO₄ pH 7, 10 mM MgCl₂ and 0.1% Triton X-100), which allowed the proteins to refold to their native state. Finally, the gel was stained for 20 min in 0.5 μg/ml ethidium bromide and destained for 20 min in the zymogram renaturation buffer. After staining with ethidium bromide, the enzymes with nuclease activity were visible as non-fluorescent bands using an UV transilluminator. The impact of boiling on the renaturing capacity of Gp5 was tested by loading both preboiled (10 min, 95° C.) and non-preboiled samples. Clear bands were observed around 8-9 kDa, indicating the nuclease activity of Gp5 (MW=8.5 KDa) (for details see FIG. 4). As no difference was observed between the preboiled and non-preboiled samples, it can be concluded that boiling does not reduce the renaturing capacity of the protein to an active enzyme. In addition, small bands at around 17 kDa were observed in each sample, indicating that Gp5 forms dimers. Despite the small fraction of Gp5 that is present as a dimer on the zymogram, this dimer:monomer ratio is not necessarily present in vivo. After all, SDS is an anionic surfactant, negatively charging proteins to allow a separation based on the molecular weight in SDS-PAGE. This step generally involves also the disintegration of dimers into monomers. The removal of SDS in the renaturation step, might allow Gp5 to reassociate into dimers.

FIG. 4 shows a zymogram, that was prepared using an SDS-PAGE gel in which 100 μg/ml DNA from fish sperm was embedded. Two concentrations of Gp5 (4.86 and 8.74 μM) were loaded and both preboiled and non-preboiled samples were used. Clear bands were observed around 8-9 kDa in all samples, indicating the presence of Gp5. Moreover, vague bands were observed around 17 kDa in both non-preboiled and preboiled samples with 8.74 μM Gp5, indicating the presence of dimers of Gp5. The protein concentrations are shown on the top. Reference: PageRuler™ Prestained Protein Ladder. This picture was enhanced to improve the visibility of the results.

### Example 3: pH Stability of P. aeruginosa Phage Protein LUZ19_Gp5

The effect of the pH on enzymatic stability and activity was tested over a pH range of 3 to 10, using a Tris/HCl buffer system. First, 10 μM of the purified LUZ19_gp5 was pre-incubated in a buffer mixture containing DNase I buffer+MgCl₂ and a Tris/HCl solution to obtain the appropriate pH, for 30 min at 37° C. Then, 100 ng of P. aeruginosa genome was added and the reaction mixtures were incubated for another 30 min at 37° C. Finally, agarose gel electrophoresis and ethidium bromide staining were used to detect the loss of nucleic acids at every pH tested. The results showed that DNase I was found to be inactive in acidic solutions, in contrast to LUZ19_Gp5 which showed enzymatic activity in all solutions tested (FIG. 5).

FIG. 5 shows an agarose gel electrophoresis based nuclease activity assay of the commercial DNase I (B) and the untagged LUZ19_Gp5 (C) incubated with 100 ng P. aeruginosa genome in various pH buffers (pH 3 to 10) for 30 min at 37° C. compared to the same reactions in absence of a nuclease (A). The proteins were first pre-incubated for 30 min in corresponding buffer to evaluate their pH stability. The pH are shown on the top. The experiment was conducted in triplicate.

### Example 4: Thermostability of P. aeruginosa Phage Protein LUZ19_Gp5

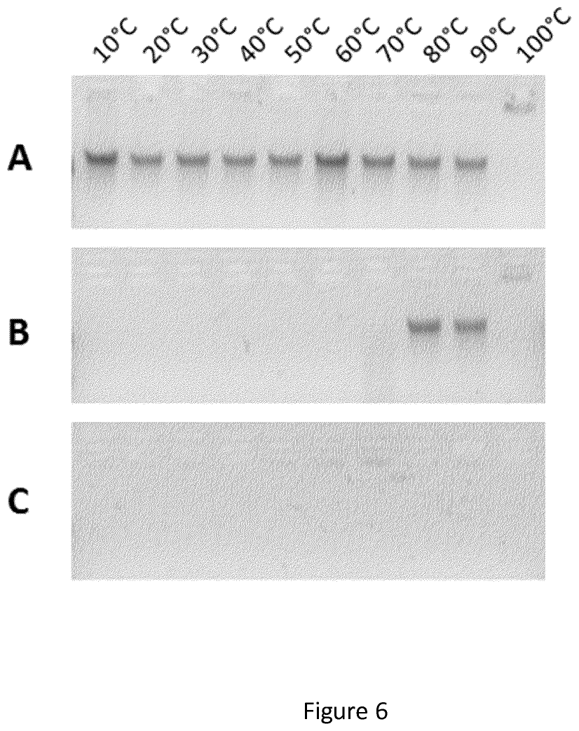

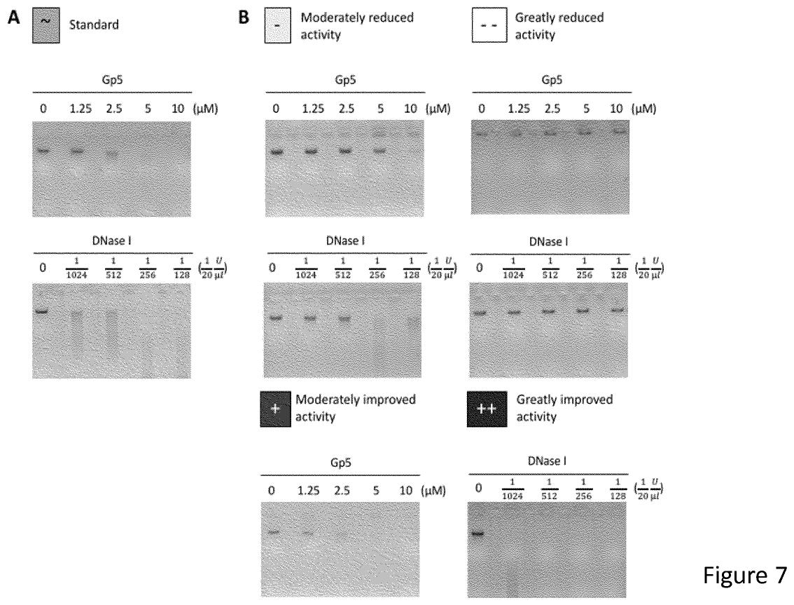

To evaluate the thermostability of the nuclease, the purified LUZ19_Gp5 protein was first pre-incubated for 30 min at various temperatures ranging from 10° C. to 100° C. in the absence of DNA, followed by incubation of the nuclease with DNA for 30 min at the corresponding temperature. Each reaction mixture consisted of 10 μM of the purified LUZ19_Gp5 protein in 20 μl DNase I buffer+MgCl₂. 100 ng of P. aeruginosa genome was added after 30 min of pre-incubation. As a negative and a positive control, milliQ water and the commercial DNase I were used respectively. In addition to the inactivation of DNase I at a temperature of 80° C., LUZ19_Gp5 remained active after incubation at every temperature tested. This demonstrated its high thermostability (FIG. 6).

Even the DNA which has been treated at 100° C. and tends to aggregates, since it remains in the loading well (FIG. 6A, 6B right lane), has been degraded by the Gp5 nuclease since no DNA is present in the right lane of FIG. 6B.

FIG. 6 shows Agarose gel electrophoresis based nuclease activity assay of the commercial DNase I (B) and the untagged LUZ19_Gp5 (C) incubated with 100 ng P. aeruginosa genome in 20 μl DNase I buffer+MgCl₂ for 30 min at various temperatures ranging from 10° C. to 100° C. compared to the same reactions in absence of a nuclease (A). The proteins were first pre-incubated for 30 min at corresponding temperature to evaluate their thermostability. The (pre-)incubation temperatures are shown on the top. The experiment was conducted in triplicate.

### Example 5: Dependence of the Nuclease Activity of the Phage-Encoded Nuclease LUZ19_Gp5 on Metal Ions and Responses to Additives. Comparison of the Nuclease Activity with the Commercial Available Nuclease DNase I

Knowing that LUZ19_Gp5 displays a substrate specificity similar to DNase I, the similarities and differences between Gp5 and DNase I are of interest. For this, agarose gel electrophoresis and ethidium bromide staining were used to detect the loss of nucleic acids under different conditions in presence of Gp5 or DNase I. Each test started with a pre-incubation period of the nuclease in DNase I buffer+MgCl₂ for 1 h at 37° C. (unless stated differently) before the substrate 5 ng/μl λ DNA (Thermo Scientific) was added. After addition, the samples were incubated another 1 h at 37° C. (unless stated differently) and then loaded on an 1% agarose gel.

The commercially available DNase I from Thermo Scientific is quantified in Units (U), while the amount of LUZ19_Gp5 that match 1 U has not been determined yet. 1 U is defined as the amount of DNase I that completely degrades 1 μg plasmid DNA in 10 min at 37° C. (Thermo Scientific). This makes it difficult to quantitatively compare both enzymes. Therefore, a qualitative method was designed to compare the activity of Gp5 to DNase I under different conditions. First, a series of four two-fold dilutions was prepared for each nuclease and an activity test was performed under standard conditions, in a way that the degradation of 5 ng/μl λ DNA was completed at the second dilution (5 μM for Gp5 and 1/256 U/20 μl for DNase I, see FIG. 7). In this way, the effect of a certain condition on the nuclease activity could be derived from the dilution at which all the DNA is cut. Next, a scoring system was designed to qualitatively determine the impact of a certain condition on the activity of the nuclease (FIG. 7).

FIG. 7 shows the scoring system used for nuclease activity tests. Agarose gel electrophoresis tests were performed under different conditions to determine the effect of a certain condition on the activity of Gp5 and DNase I. A) A visualization of the activity of Gp5 and DNase I under standard conditions (pre-incubation without DNA: 1 h, 37° C.; incubation: 1 h, 37° C., 5 ng/μl λ DNA, DNase I buffer+MgCl₂, no additives and pH 7.5). B) Visualization of the scoring system for the nuclease activity under a specific condition compared to the standard condition. If no picture is present for a certain enzyme in combination with a certain score, it means that no results were obtained for this combination.

After developing the scoring system, the nuclease activity of both Gp5 and DNase I was evaluated under various experimental conditions. These conditions were obtained by varying the (pre)incubation temperature and time, changing the pH of the samples or adding different compounds to the samples, like metal chelators, reducing agents, denaturants, detergents and ions. To improve the reliability, each activity test was done in triplicate. The results of the activity tests under the different conditions are summarized in table 4.

TABLE 4
Evaluation of the activity of LUZ19_Gp5 and DNase I under different conditions.

| | Conditions | Gp5 | | | DNase I | | |
|---|---|---|---|---|---|---|---|
| Temperature | 10° C. | ~ | ~ | ~ | ~ | ~ | No data |
| (5 h pre- | 90° C. | ~ | ~ | ~ | − − | − − | − − |
| incubation) | 99.9° C. | − | − | − | − − | − − | − − |
| pH | pH 1 | − | − | ~ | − − | − − | − − |
| | pH 7 | ~ | ~ | ~ | − − | − − | − − |
| | pH 10 | + | + | + | − | − | − |
| Metal ions | No supplemented | − | − | − | − − | − − | − − |
| (buffer w/o | ions | | | | | | |
| MgCl2) | 10 mM MgCl₂ | ~ | ~ | ~ | ~ | ~ | − |
| | 10 mM MnCl₂ | ~ | − | − | ~ | ~ | ~ |
| | 10 mM CaCl₂ | ~ | ~ | ~ | − − | − − | − − |
| Metal chelator | 10 mM EDTA | ~ | ~ | ~ | − − | − − | − − |
| | 1 mM citrate | ~ | ~ | ~ | − | − | − |
| | 20 mM citrate | − − | − − | − − | − − | − − | − − |
| | 200 mM citrate | − − | − − | − − | − − | − − | − − |
| Ionic strength | 70 mM sodium | − − | − − | − − | − − | − − | − − |
| | phosphate | | | | | | |
| Reducing | 100 mM | ~ | ~ | ~ | − − | − − | − − |
| agent | Dithiothreitol | | | | | | |
| | (DTT) | | | | | | |
| | 1% (v/v) β- | − | − | − | − | − | − |
| | mercaptoethanol | | | | | | |
| | (143 mM) | | | | | | |
| Denaturing | 200 mM guanidine | ~ | ~ | ~ | − − | − − | − − |
| agents | hydrochloride | | | | | | |
| | 2M guanidine | − − | − − | − − | − − | − − | − − |
| | hydrochloride | | | | | | |
| Detergent | 1% (v/v) Tween- | − | − | − | ++ | ++ | ++ |
| | 20 | | | | | | |

Activity tests were performed under different conditions for both Gp5 and DNase I. Each test was done in triplicate to improve the reliability of the results. The results of each test were scored according to the developed scoring system (FIG. 7).
Different symbols were assigned to a certain level of activity; [~] indicates that the activity of the nuclease under a certain condition is similar to the standard, [−] indicates a moderately reduced activity compared to the standard, [− −] indicates a greatly reduced activity, [+] indicates a moderately improved activity and [++] indicates a greatly improved activity.
The above results indicate that the Gp5 nuclease is more active than DNase I in a variety of conditions, with the exception of non-ionic detergents.

The possibility of protein refolding after heat treatment was considered as well, as some heat denatured proteins can refold to their active conformation when cooled down slowly. To investigate the possibility that LUZ19_Gp5 is reversibly denatured at 90° C., an additional nuclease activity test in which the samples were cooled down rapidly on ice followed by the addition of the Gp5 inhibitor citrate (final concentration: 20 mM, see Table 4: chelators) after 10 seconds was performed. The results indicate that Gp5 retains its capacity to degrade the DNA under these conditions, which indicates that Gp5 is not denatured at 90° C. and nuclease activity is still effective at high temperatures.

### Example 6: Recombinant Expression of P. aeruginosa Phage Protein LUZ19_Gp5 in P. aeruginosa. Cloning and Expression in P. aeruginosa

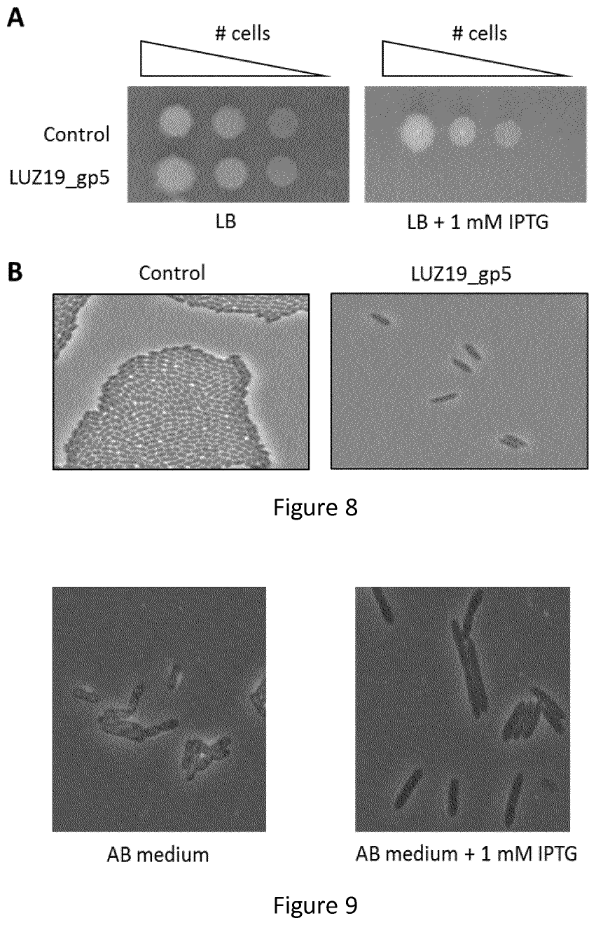

LUZ19_gp5 was amplified with Pfu polymerase using specific primers (Table 5) and phage LUZ19 genome as a template, and cloned in a Gateway entry vector using the pENTR/SD/D-TOPO cloning kit (Invitrogen) according to the manufacturer's protocol. The gene was then transferred to the E. coli-P. aeruginosa shuttle expression vector pUC18-mini-Tn7T-Lac, which first was made Gateway compatible, using the Gateway® LR Clonase® enzyme mix (Thermo Scientific) following the protocol provided by the manufacturer. Co-transformation of 300 ng of the pUC18-mini-Tn7T-Lac-GW construct and 500 ng pTNS2 plasmid by electroporation to P. aeruginosa PAO1 allowed single-copy integration of the phage gene in the Pseudomonas genome under the control of an IPTG-inducible lac promotor. Finally, three 100-fold dilution series (10⁰, 10⁻², 10⁻⁴ and 10⁻⁶), each of an independent overnight culture, were spotted in parallel on LB medium (1% (w/v) Bacto tryptone, 1% (w/v) NaCl and 0.5% (w/v) yeast extract) with and without 1 mM IPTG. As a negative control, a P. aeruginosa PAO1 strain, encoding an empty pUC18-mini-Tn7T-Lac-GW expression cassette, was used (FIG. 8A).

TABLE 5
Primers used during PCR amplification of
LUZ19_gp5 for Gateway cloning
into the pENTR/SD/D-TOPO vector

| | | |
|---|---|---|
| LUZ19_gp5 | Primer 1 | CACCATGTCCTCGCGTGATCCC |
| | | [SEQ ID NO: 30] |
| | Primer 2 | TCACCGCTCCGCCCGCTG |
| | | [SEQ ID NO: 31] |

To investigate the effect on the host morphology, the cell growth of the P. aeruginosa PAO1 cells with the phage gene integrated in their genome was analyzed over time using time-lapse microscopy. For this, overnight cultures were prepared in LB medium supplemented with 30 μg/ml Gentamicin (Gm³⁰; VWR international Ltd). Next, 2 μl of a thousand-fold dilution of the overnight culture was spotted on LB agar (LB with 1.5% (w/v) agar) supplemented with 1 mM IPTG. Growth was recorded at 37° C. (Okolab Ottaviano, Italy) in real time for 5 h with a Ti-Eclipse inverted microscope (Nikon, Champi-gny-sur-Marne, France) connected to a Nikon DS-Qi2 camera (FIG. 8B).

FIG. 8 shows the phenotypic effect of LUZ19_gp5 expression on P. aeruginosa PAO1 growth. (A) A serial dilution of P. aeruginosa PAO1 cells, containing single-copy integration of the LUZ19_gp5 gene under control of an IPTG-inducible promotor, was spotted on solid LB media with (right) and without (left) 1 mM IPTG. As a negative control, a P. aeruginosa PAO1 strain, encoding an empty pUC18-mini-Tn7T-Lac expression cassette, was used. No growth of the mutant strain with integrated LUZ19_gp5 gene was detected in presence of IPTG, indicating that Gp5 has an antibacterial (toxic) effect on the growth of PAO1. (B) Microscopic recording of P. aeruginosa cells with (right) and without (left) integrated LUZ19_gp5 gene grown for 5 h in presence of 1 mM IPTG. LUZ19_gp5 expression leads to growth arrest of P. aeruginosa PAO1. Furthermore, after five hours of expression, the cells have become slightly larger (about 2 times the original size).

### Example 7: Determination of In Vivo Nuclease Activity of P. aeruginosa Phage Protein LUZ19_Gp5

The in vivo nuclease activity of LUZ19_Gp5 was examined under a microscope using the P. aeruginosa PAO1 cells with the LUZ19_gp5 gene integrated in their genome (see example 6) and SYBR Green I to stain the P. aeruginosa genome.

First, two overnight cultures were prepared in LB medium supplemented with Gm³⁰. The next day, 80 μl of these cultures were transferred to fresh 4 ml LB medium supplemented with Gm³⁰ and the cells were grown at 37° C. until an OD_{600 nm}of 0.2 was reached. Then, 1 mM IPTG was added two one culture and both cultures were incubated for 2 h at 37° C. before 0.01% (v/v) SYBR Green I was added to both samples. The samples were incubated 20 min while shaking, and 2 μl of each sample was spotted on AB medium (3 mM (NH₄)₂SO₄, 18 mM Na₂HPO₄, 4 mM KH₂PO₄, 10 mM NaCl, 0,1 mM CaCl₂, 1 mM MgCl₂ and 3 μM FeCl₃). Microscopic images were taken with a Ti-Eclipse inverted microscope connected to a Nikon DS-Qi2 camera.

FIG. 9 shows visualization of the in vivo nuclease activity of LUZ19_Gp5 in P. aeruginosa. Microscopic recordings of P. aeruginosa cells with integrated LUZ19_gp5 gene without (left) and after 2 h induction (right) with 1 mM IPTG. The P. aeruginosa genome was stained with 0.01% (v/v) SYBR Green I (grey speckles on left panel). The cells without expression of LUZ19_Gp5 contained green clouds, owing to the staining of the bacterial DNA with SYBR Green I. After expression of the phage protein the P. aeruginosa genome was not stained anymore. Instead, elongated cells without green clouds were detected. This observation confirms the nuclease activity of LUZ19_Gp5 in vivo, as no DNA-dye complex was detected due to the degradation of the P. aeruginosa genome after expression of the phage protein.

### Example 8: Loss-of-Toxicity Mutants of P. aeruginosa Phage Protein LUZ19_Gp5. Identification, Cloning, Recombinant Expression in P. aeruginosa, Purification and Determination of Biochemical Nuclease Activity of LUZ19_Gp5 Non-Toxic Mutants

The toxicity of LUZ19_Gp5 prevents bacterial growth. However, the presence of a mutation in the gene can result in loss of toxicity. Such mutations were identified by plating P. aeruginosa PAO1 cells containing the pUC18-mini-Tn7T-Lac-GW expression cassette with LUZ19_gp5 gene inserted in their genome overnight on LB with 1 mM IPTG. Next, the pUC18-mini-Tn7T-Lac-GW cassettes of present colonies were amplified using vector primers (Table 6, primers 1-2) and the sequences of both promotor region and the phage gene were determined.

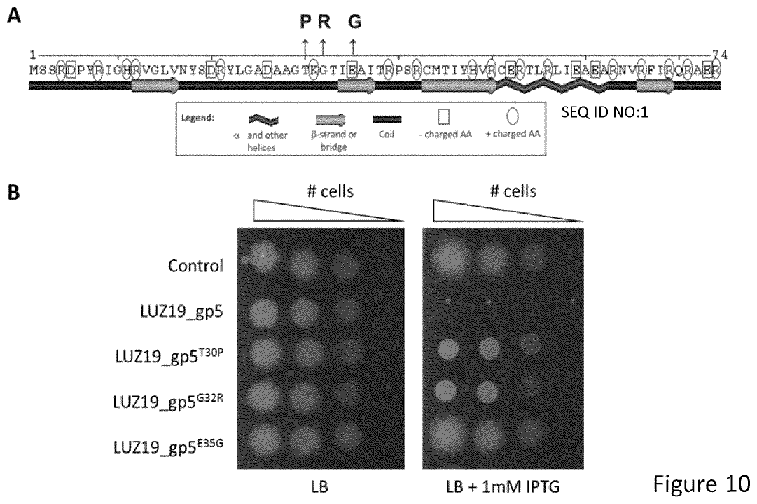

Three different mutations were identified in the LUZ19_gp5 gene potentially neutralizing its toxicity (FIG. 10A). Subsequently, three alternative pUC18-mini-Tn7T-Lac-GW plasmids were constructed by site-directed mutagenesis, each containing the phage gene with another mutation, to confirm that indeed the identified mutations resulted in a loss of toxicity. First, primers were designed that contain the desired mutation and enable amplification of the plasmid (Table 6, primers 3-8). These primers were phosphorylated by adding 4 μl T4 polynucleotide kinase (Thermo Scientific) and 1 mM dATP (Thermo Scientific) to 4 μl 100 μM primer in reaction buffer A (Thermo Scientifc) and incubation for 1 h at 37° C., followed by inactivation for 10 min at 70° C. The phosphorylated primers were then used in a PCR reaction with Phusion High-Fidelity polymerase to introduce the mutation. For this, 9 pg of the original pUC18-mini-Tn7T-Lac-GW plasmid with integrated LUZ19_gp5 gene was incubated with 1 U Phusion High-Fidelity polymerase (Thermo Scientific), 0.2 mM dNTPs (Thermo scientific) and 0.6 μM forward and reverse primer in Phusion GC buffer (Thermo Scientific) using following parameters:

Site-directed mutagenesis
(Phusion polymerase)

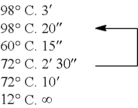

The obtained PCR fragments were purified using the GeneJet™ Gel extraction kit (Thermo Scientific) according to the manufacturer's protocol. 25 ng of the purified PCR mix was then incubated with 0.5 μl Quick T4 ligase (New England BioLabs) in the corresponding buffer following the protocol provided by the manufacturer. If the sequence of the obtained plasmid was correct, a co-transformation of 300 ng of this pUC18-mini-Tn7T-Lac-GW construct and 500 ng pTNS2 was performed that allow single-copy integration of the mutated phage gene in the Pseudomonas genome. Finally, three 100-fold dilution series (10⁰, 10⁻², 10⁻⁴ and 10⁻⁶), each of an independent overnight culture, were spotted in parallel on LB medium with and without 1 mM IPTG. As a negative and positive control, a P. aeruginosa PAO1 strain encoding an empty pUC18-mini-Tn7T-Lac-GW expression cassette and one with integrated wild-type LUZ19_gp5 gene were used respectively (FIG. 10B).

TABLE 6
Primers used for the identification of LUZ19_Gp5 non-toxic
mutants (primers 1-2) and the construction of
pUC18-mini-Tn7T-Lac-GW plasmids, containing the phage gene
with a single mutation, by site-directed mutagenesis (primers 3-8).

| | | |
|---|---|---|
| pUC18-mini- | Primer 1 | ATCATGCCATACCGCGAAAGGTTTTGCACCA [SEQ ID NO: 32] |
| Tn7T-Lac-GW | | GGAGGGGTGGAAATGGAGTT [SEQ ID NO: 33] |
| | Primer 2 | |
| LUZ19_gp5^{T30P} | Primer 3 | GCACCATCGGAGCCATAACCC [SEQ ID NO: 34] |
| | Primer 4 | CCTTGGTGCCTGCCGC [SEQ ID NO: 35] |
| LUZ19_gp5^{G32R} | Primer 5 | CGGCAGGCCCCAAGGGCAC [SEQ ID NO: 36] |
| | Primer 6 | CGTCGGCACCCAGGTAGCGGTCG [SEQ ID NO: 37] |
| LUZ19_gp5^{E35G} | Primer 7 | CATAACCCGACCGTCCCGCTG [SEQ ID NO: 38] |
| | Primer 8 | GCTTCGATGGTTCTCTTGGTGCCTGC [SEQ ID NO: 39] |

The underlined bases indicate mutations compared to the origiinal sequence.

FIG. 10 shows an overview of the identified mutations that abolish toxicity upon introduction in LUZ19_Gp5. (A) The amino acid sequence of Gp5 and its missense mutants resulting in a loss-of-toxicity towards PAO1. Negatively charged amino acids are indicated in a rectangle, positively charged amino acids are indicated in an oval. Secondary structures were predicted with ‘Sable secondary prediction server’. Mutants from left to right: LUZ19_Gp5^{T30P}, LUZ19_Gp5^{G32R} and LUZ19_Gp5^{E35G}. (B) Serial dilutions (10⁰, 10⁻², 10⁻⁴ and 10⁻⁶) of P. aeruginosa PAO1 cells, containing single-copy integration of the (mutated) LUZ19_gp5 gene under control of an IPTG-inducible promotor, were spotted on solid LB media with (right) and without (left) 1 mM IPTG. As a negative control, a P. aeruginosa PAO1 strain, encoding an empty pUC18-mini-Tn7T-Lac expression cassette, was used. Next, the three non-toxic mutant proteins of LUZ19_Gp5 were studied to evaluate the impact of each mutation on the activity of this enzyme. First, the expression plasmids were constructed by site-directed mutagenesis similar to the construction of the pUC18-mini-Tn7T-Lac-GW plasmids with mutated phage gene, using the same primers (table 6, primers 3-8) and the pGEX-6P-1 vector containing the LUZ19_gp5 gene as a template. Recombinant expression and purification of the three mutant proteins was performed identical to the purification of the wild-type protein (See example 1).

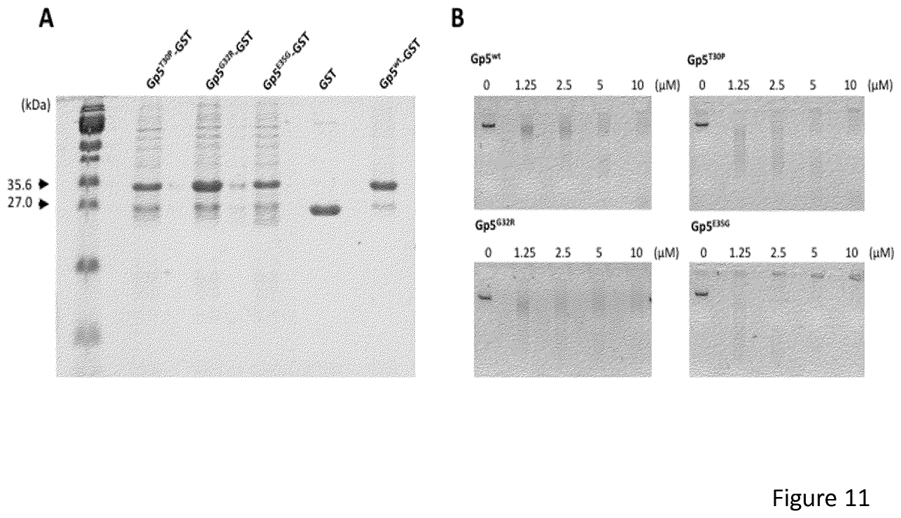

Since size exclusion chromatography failed for each mutant, the mutant proteins fused to a GST-tag were selected for the nuclease tests. Purified stock solutions of the three mutant proteins were analyzed visually on an 16% SDS-PAGE gel (FIG. 11A). Finally, an agarose gel electrophoresis-based nuclease activity test was performed in triplicate for each mutant protein under the standard conditions defined in example 5 (FIG. 11B).

FIG. 11 shows nuclease activity of the non-toxic LUZ19_Gp5 mutants. (A) The composition of the different samples used for the mutant nuclease tests, visualized by SDS-PAGE. The GST-tagged LUZ19_Gp5 (Gp5-GST) fusion proteins are 35.6 kDa and the GST-tag is 27.0 kDa. (B) Agarose gel electrophoresis based nuclease activity tests performed with wild-type LUZ19_Gp5-GST (Gp5^{wt}) and the three GST-tagged non-toxic mutants (Gp5^{T30P}, Gp5^{G32R} and Gp5^{E35G}). Each test was performed under standard conditions.

### Example 9: In Silico Analysis of P. aeruginosa Phage Protein LUZ19_Gp5. The Search for Homologs on Nucleic Acid and Amino Acid Level

The Basic Local Alignment Search Tool (BLAST) was used to find homologous proteins of LUZ19_Gp5. Both the nucleotide and protein sequence were compared to sequence databases using the standard nucleotide BLAST (blastn) and the translated BLAST (tblastx), and the results are shown in Table 7 and Table 8.

TABLE 7
Results of blastn search for homologs of LUZ19_gp5. The homologous genes are
listed in column one, all belonging to Pseudomonas phages of the Podoviridae family
(column three). The second column shows the length of the identified genes in base
pairs (bp), and columns four, five and six indicate the statistical significance
of the matches. The last column shows the accession number of each homolog.

| | Length | | Query | | Identity | Accession |
|---|---|---|---|---|---|---|
| Description | (bp) | Genus^{a} | cover | E value | % | number |
| Phage LUZ19 | 225 | Phikmvvirus | 100% | 1e−112 | 100 | AM910651.1 |
| gp5 | | | | | | |
| Phage DL62 | 228 | Phikmvvirus | 93% | 1e−96 | 98 | KR054031.1 |
| Phage MPK6 | 225 | Phikmvvirus | 100% | 2e−94 | 95 | JX997978.1 |
| ORF5 | | | | | | |
| Phage | 228 | Phikmvvirus | 93% | 1e−91 | 96 | KM067278.1 |
| vB_PaeP_PPA-ABTNL ORF7 | | | | | | |
| Phage | 228 | unclassified | 93% | 1e−86 | 95 | LN610574.1 |
| vB_PaeP_PAO1_Ab05 ORF6 | | | | | | |
| Phage MPK7 | 228 | Phikmvvirus | 93% | 1e−86 | 95 | JX501340.1 |
| ORF7 | | | | | | |
| Phage phikF77 | 228 | Phikmvvirus | 93% | 1e−86 | 95 | FN263372.1 |
| ORF5 | | | | | | |
| Phage phiNFS | 228 | Phikmvvirus | 93% | 1e−81 | 93 | KU743887.1 |
| gp6 | | | | | | |
| Phage | 372 | Phikmvvirus | 93% | 1e−81 | 93 | LN610580.1 |
| vB_PaeP_PAO1_1-15pyo ORF6 | | | | | | |
| Phage vB_Pae- | 228 | Phikmvvirus | 93% | 1e−81 | 93 | JQ307386.1 |
| TbilisiM32 gp5 | | | | | | |
| Phage PT5 | 228 | Phikmvvirus | 93% | 1e−81 | 93 | EU056923.1 |
| Phage PT2 | 228 | T7virus | 90% | 6e−46 | 84 | EU236438.1 |
| Phage phiKMV | 228 | Phikmvviru | 90% | 6e−46 | 84 | AJ505558.1 |
| ORF5 | | s | | | | |
| Phage LKD16 | 219 | Phikmvvirus | 70% | 9e−21 | 75 | AM265638.1 |
| ORF5 | | | | | | |

^{a}genera belonging to the Podoviridae family. All these viruses have P. aeruginosa as host

TABLE 8
Results of tblastx search for homologs of LUZ19_Gp5. The homologous proteins
are listed in column one, all belonging to Pseudomonas phages of the Podoviridae
family (column four). The second and third columns shows the molecular weight (MW)
in kilodaltons (kDa) and the theoretical pI of the identified protein calculated
with ExPASy ProtParam, and column five indicates the statistical significance of
the matches. The last column shows the accession number of each homolog.

| | MW | | | | |
|---|---|---|---|---|---|
| Description | (kDa) | pI | Genus | Ident | Accession number |
| Phage LUZ19 gp5 | 8.58 | 10.45 | Phikmvvirus | 100% | YP_001671947.1 |
| Phage MPK6 gp5 | 8.58 | 10.45 | Phikmvvirus | 97% | YP_008766772.1 |
| Phage | 8.68 | 10.53 | Phikmvvirus | 95% | AIK67568.1 |
| vB_PaeP_PPA- | | | | | |
| ABTNL gp7 | | | | | |
| Phage | 8.68 | 10.53 | unclassified | 95% | YP_009125704.1 |
| vB_PaeP_PAO1_Ab | | | | | |
| 05 gp6 | | | | | |
| Phage | 13.75 | 11.15 | Phikmvvirus | 93% | CEF89885.1 |
| vB_PaeP_PA01_{—} | | | | | |
| 1-15pyo gp6 | | | | | |
| Phage vB_Pae- | 8.66 | 10.26 | Phikmvvirus | 93% | YP_006299927.1 |
| TbilisiM32 gp5 | | | | | |
| Phage DL62 gp8 | 8.59 | 10.82 | Phikmvvirus | 93% | YP_009201863.1 |
| Phage MPK7 gp7 | 8.66 | 10.53 | Phikmvvirus | 93% | YP_008431315.1 |
| Phage phikF77 gp6 | 8.71 | 10.76 | Phikmvvirus | 93% | YP_002727825.1 |
| Phage phiNFS gp6 | 8.66 | 10.26 | Phikmvvirus | 93% | AMQ66144.1 |
| Phage PT5 gp5 | 8.66 | 10.26 | Phikmvvirus | 93% | ABW23084.1 |
| Phage PT2 gp5 | 8.74 | 9.44 | T7virus | 78% | ABY70972.1 |
| Phage phiKMV gp5 | 8.74 | 9.44 | Phikmvvirus | 78% | NP_877444.1 |
| Phage LKD16 gp5 | 8.38 | 9.73 | Phikmvvirus | 72% | YP_001522794.1 |

### Example 10: Determination of the Biochemical Nuclease Activity of a Homologous Protein of LUZ19_Gp5. Cloning, Recombinant Expression in E. coli, Purification and Determination of the Nuclease Activity of phiKMV_Gp5

One homolog of LUZ19_Gp5, namely phiKMV_Gp5 [SEQ ID NO: 8], was selected and tested on nuclease activity. Compared to LUZ19_Gp5, this protein has an E-value of 4e-36 and 78% of the amino acids are identical between both proteins (table 8).

First, the open reading frame (ORF5) encoding the hypothetical protein phiKMV_Gp5 was directionally cloned into a pGEX-6P-1 vector similar to the cloning of LUZ19_gp5 gene into the same vector (see example 1). The used primers for PCR are shown in Table 9.

TABLE 9
Primers used during PCR amplification of
phiKMV_gp5. The underlined bases indicate the
restriction sites of restriction enzymes
BamHI and EcoRI respectively.

| | | |
|---|---|---|
| N-terminal | Primer 1 | ATAGGATCCATGTACTCGCATGACC |
| GST-tag | | [SEQ ID NO: 40] |
| | Primer 2 | ATAGAATTCTCATTTACGGGTCGGC |
| | | [SEQ ID NO: 41] |

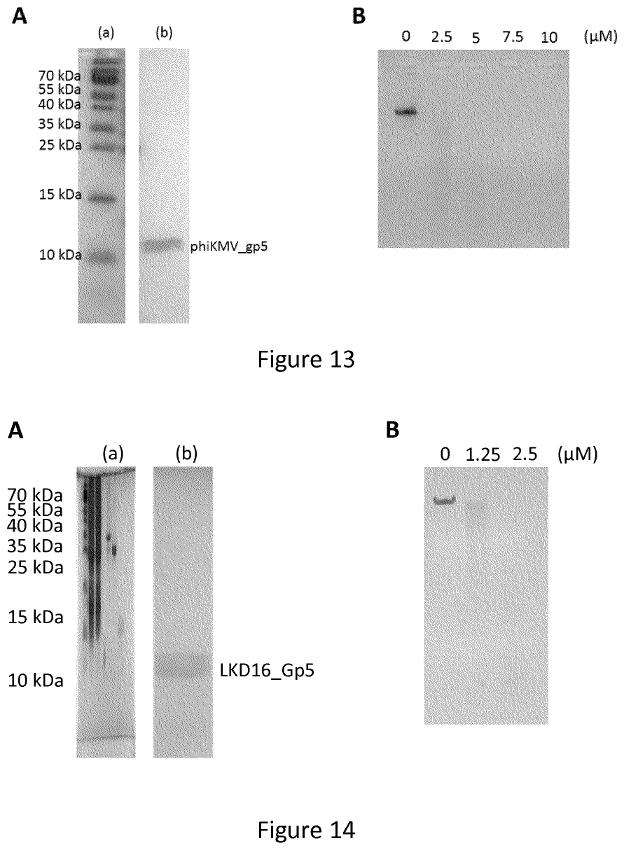

Recombinant expression and purification of the protein was performed similar to the purification of the LUZ19_Gp5 protein (see example 1). The yields after affinity-based purification (N-terminal GST-tag), and removal of the GST-tag followed by size-exclusion chromatography (untagged) are shown in Table 10. The protein concentration was determined spectrophotometrically at a wavelength of 280 nm. The purified stock solution of the protein was analyzed visually on an 16% SDS-PAGE gel (FIG. 13A).

TABLE 10
Yields of purified recombinant phiKMV_Gp5 protein
per liter E. coli expression culture as determined
by spectrophotometric measurement at 280 nm

| phiKMV_Gp5 | Expression yield | |
|---|---|---|
| N-terminal GST-tag | 9 | mg |
| Untagged | 0.2 | mg |

The nuclease activity of phiKMV_Gp5 was studied similar to the nuclease activity of LUZ19_Gp5 (See example 5). The test started with a pre-incubation period of the nuclease in DNase I buffer+MgCl₂ for 1 h at 37° C. before the substrate 5 ng/μl λ DNA was added. After addition, the samples were incubated another 1 h at 37° C. and then loaded on an 1% agarose gel. The result showed that DNA was completely degraded, even at the lowest concentration tested (FIG. 13B).

FIG. 11 shows the nuclease activity of phiKMV_Gp5. (A) SDS-PAGE analysis of the purified phiKMV_Gp5 protein (lane a) alongside a PageRuler™ prestained protein ladder (lane b). (B) Agarose gel electrophoresis based nuclease activity test performed with phiKMV_Gp5. The protein was first pre-incubated for 1 h at 37° C., followed by an incubation step with 100 ng λ DNA of 1 h at 37° C. The protein concentrations are shown on the top.

### Example 11 Comparative Data of DNases

Table 11 compares the properties of the miniDNase of the present invention with those of commercially available products.

TABLE 11
properties of various DNAses

| | | | DNA | DNA | | | | | | | | |
|---|---|---|---|---|---|---|---|---|---|---|---|---|
| Commercial | Endo- | Exo- | single | double | | | | Temp | | | | Ion- |
| nucleases | nuclease | nuclease | strand | strand | RNA | MW | | (optimal) | | pH (optimal) | | dependent |
| miniDNases of the | X | Tba | X | X | | <10 | kDa | <100° | C. | 1-10 | (10) | Mg²⁺ or Ca²⁺ |
| invention | | | | | | | | | | | | |
| Dnase I | X | | X | X | | ~31 | kDa | <70° | C. | 6-10 | (7-8) | Divalent cation |
| bovine pancreas | | | | | | | | | | | | (Mg²⁺) |
| Sigma-Aldrich | | | | | | | | | | | | |
| Dnase II | X | | X | X | | 38 | kDa | | | 4-6.5 | (5) | |
| bovine spleen | | | | | | | | | | | | |
| Sigma-Aldrich | | | | | | | | | | | | |
| Benzonase EMD | X | | X | X | X | ~30 | kDa | 0-42° | C. | 6-10 | (8-9.2) | Mg²⁺ |
| Millipore | | | | | | | | (37° | C.) | | | |
| Micrococcal Nuclease | X | X | X | X | X | ~17 | kDa | (28° | C.) | (9-10) | | Ca²⁺ |
| S. aureus | | | | | | | | | | | | |
| Sigma-Aldrich | | | | | | | | | | | | |
| Nuclease P1 | | | X | | X | 42-50 | kDa | (70° | C.) | 5-8 | | Zn²⁺ |
| P. citrinum | | | | | | | | | | | | |
| Sigma-Aldrich | | | | | | | | | | | | |
| Nuclease S1 | X | X | X | | X | ~34 | kDa | (37° | C.) | (4-4.6) | | Ca²⁺ or Zn²⁺ |
| A. oryzae | | | | | | | | | | | | |
| Sigma-Aldrich | | | | | | | | | | | | |
| RNAse A bovine | X | | | | X | ~14 | kDa | 15-70° | C. | 6-10 | (7.6) | |
| pancreas | | | | | | | | (60° | C.) | | | |
| Dornase alpha | X | | X | X | | ~37 | kDa | | | | | Divalent cation |
| (=Dnase I) | | | | | | | | | | | | |
| (Pulmozyme) | | | | | | | | | | | | |

### Example 12: Determination of the Biochemical Nuclease Activity of a Homologous Protein of LUZ19_Gp5. Cloning, Recombinant Expression in E. coli, Purification and Determination of the Nuclease Activity of LKD16_Gp5

Besides phiKMV_Gp5, an additional similar protein of LUZ19_Gp5, namely LKD16_Gp5, was tested for nuclease activity. This protein has the lowest blast similarity to LUZ19_Gp5 of all available proteins (Table 8) with an E-value of 1e-25 and 72% identical amino acids between both proteins (table 8). Furthermore, LKD16_Gp5 (SEQ ID NO: 9) consists of only 72 amino acids, compared to LUZ19_Gp5 (SEQ ID NO: 1) and phiKMV_Gp5 (SEQ ID NO: 8) which consist of 74 and 75 amino acids respectively.

Like with phiKMV_gp5, the open reading frame (ORF5) encoding the hypothetical protein LKD16_Gp5 was directionally cloned into a pGEX-6P-1 vector similar to the cloning of LUZ19_gp5 gene into the same vector (see example 1). The used primers for PCR are shown in Table 12.

TABLE 12
Primers used during PCR
amplification of LKD16_gp5.

| | | |
|---|---|---|
| N-terminal | Primer 1 | ATAGGATCCATGTTCTTGCATGACCG |
| GST-tag | | GTTC |
| | | [SEQ ID NO: 42] |
| | Primer 2 | TATGAATTCTCAGCGCCCCAGCCAG |
| | | [SEQ ID NO: 43] |

The underlined bases indicate the restriction sites
of restriction enzymes BamHI and EcoRI respectively.

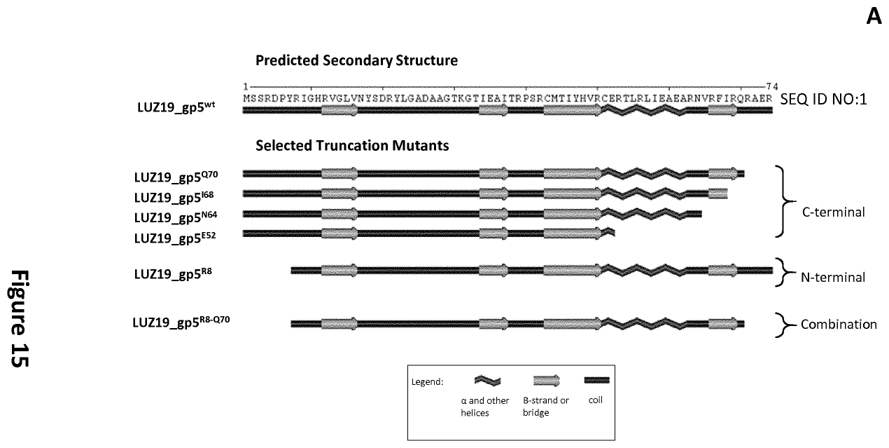

Recombinant expression and purification of the protein was performed similar to the purification of the LUZ19_Gp5 protein (see example 1). The yields after affinity-based purification (N-terminal GST-tag), and removal of the GST-tag followed by size-exclusion chromatography (untagged) are shown in Table 13. The purified stock solution of the protein was analyzed visually on a 16% SDS-PAGE gel (FIG. 14A).

TABLE 13
Yields of purified recombinant LKD16_Gp5 protein
per liter E. coli expression culture as determined
by spectrophotometric measurement at 280 nm

| phiKMV_Gp5 | Expression yield | |
|---|---|---|
| N-terminal GST-tag | 4.7 | mg |
| untagged | 0.06 | mg |

The nuclease activity of LKD16_Gp5 was analyzed similar to the nuclease activity of LUZ19_Gp5 (See example 5) and concentrations up to 2.5 μM have been tested. The DNA digestion after incubation with LKD16_Gp5 is shown in FIG. 14B.

FIG. 14 shows the nuclease activity of LKD16_Gp5. Panel A shows (A) SDS-PAGE analysis of the purified LKD16_Gp5 protein (lane a) alongside a PageRuler™ prestained protein ladder (lane b). Panel (B) shows agarose gel electrophoresis based nuclease activity test performed with LKD16_Gp5. The protein was first pre-incubated for 1 h at 37° C., followed by an incubation step with 100 ng λ DNA of 1 h at 37° C. The protein concentrations are shown above.

### Example 13: Truncation Mutants of P. aeruginosa Phage Protein LUZ19_Gp5. Selection, Cloning, Recombinant Expression in E. coil, Purification, Determination of Biochemical Nuclease Activity and Recombinant Expression in P. aeruginosa of LUZ19_Gp5 Truncation Mutants

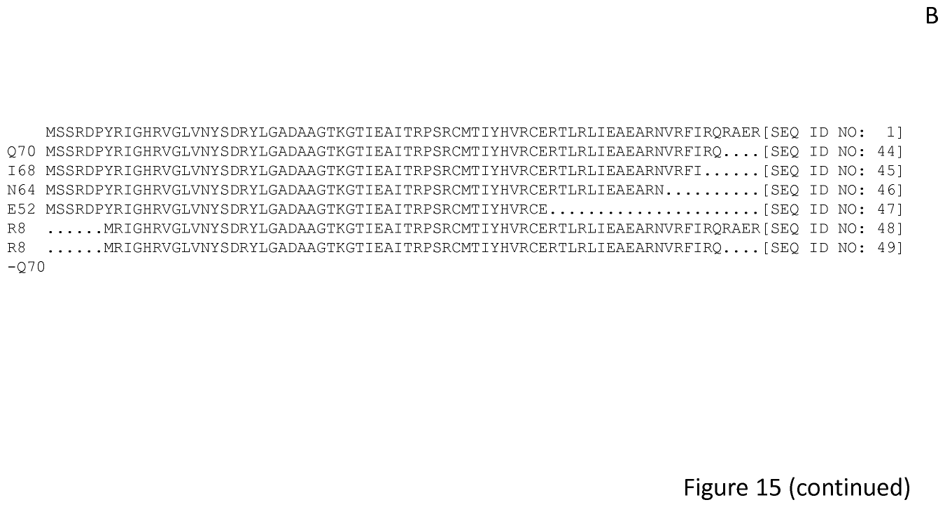

Truncation mutants are variants of LUZ19_Gp5 that have a deletion of specific amino acids at the N- and/or C-terminus of the protein. Studying their activity in vitro and in vivo gives more insights into the protein region essential for its nuclease activity and toxicity, respectively. Based on the amino acid conservation of the homologs of LUZ19_Gp5 and on the predicted secondary structure, seven truncation mutants have been selected as examples (FIG. 15 and Table 14). Their biochemical nuclease activity has been assayed in vitro and their toxicity has been evaluated in vivo by expression in P. aeruginosa PAO1.

TABLE 14
Amino acid sequences of the seven selected
LUZ19_gp5 truncation mutants.

| Truncation | | |
|---|---|---|
| terminus | Name | Protein Sequence |
| C-terminal | LUZ19-gp5^{Q70} | MSSRDPYRIGHRVGLVNYSDRYLGA |
| | | DAAGTKGTIEAITRPSRCMTIYHVR |
| | | CERTLRLIEAEARNVRFIRQ |
| | | [SEQ ID NO: 44] |
| | LUZ19_gp5^{I68} | MSSRDPYRIGHRVGLVNYSDRYLGA |
| | | DAAGTKGTIEAITRPSRCMTIYHVR |
| | | CERTLRLIEAEARNVRFI |
| | | [SEQ ID NO: 45] |
| | LUZ19_gp5^{N64} | MSSRDPYRIGHRVGLVNYSDRYLGA |
| | | DAAGTKGTIEAITRPSRCMTIYHVR |
| | | CERTLRLIEAEARN |
| | | [SEQ ID NO: 46] |
| | LUZ19_gp5^{E53} | MSSRDPYRIGHRVGLVNYSDRYLGA |
| | | DAAGTKGTIEAITRPSRCMTIYHCR |
| | | CE |
| | | [SEQ ID NO: 47] |
| N-terminal | LUZ19_gp5^{R8} | MRIGHRVGLVNYSDRYLGADAAGTK |
| | | GTIEAITRPSRCMTIYHVRCERTLR |
| | | LIEAEARNVRFIRQR |
| | | [SEQ ID NO: 48] |
| N- and C- | LUZ19_gp5^{R8-Q70} | MRIGHRVGLVNYSDRYLGADAAGTK |
| terminal | | GTIEAITRPSRCMTIYHVRCERTLR |
| | | LIEAEARNVRFIRQ |
| | | [SEQ ID NO: 49] |

To express the truncation mutants in E. coli for in vitro nuclease assessment, seven pGEX-6P-1 expression plasmids were constructed by site-directed mutagenesis, each containing the phage gene with a 5′ and/or 3′ deletion of coding nucleotides. To create N- or C-terminal truncations, the original pGEX-6P-1 plasmid expressing LUZ19_gp5_wt was used as template for the amplification (see example 1). First, primers were designed that amplify the entire pGEX-6P-1 expression plasmid, except for the corresponding 5′ or 3′ nucleotides that result in the desired truncation. For this, one primer annealed to the vector backbone right next to the 5′ or 3′ end of the coding sequence (referred to as backbone primer, Table 15A: primers 1-2), while the other primer annealed to the coding sequence (referred to as coding primers, table 15B: primers 1-6), causing a deletion of certain nucleotides. For the truncation with a deletion both at the N- and C-terminus (i.e. LUZ19_gp5^{R8-Q70}), pGEX-6P-1 expressing LUZ19_gp5^{R8} was used as template. This template was then amplified with backbone primer 1 and coding primer 1 (Table 15A and B). Primer phosphorylation, site-directed mutagenesis and PCR fragment purification were performed exactly as in example 8. Next, the truncation mutants were expressed as N-terminal GST-tagged proteins, exactly as described in example 1. If protein yields >1 mg/l were obtained, the N-terminal GST-tag was removed to obtain untagged proteins, as described in example 1. The yields for the purifications of recombinant LUZ19_Gp5 truncation mutants are shown in Table 16. The protein concentration was determined spectrophotometrically at a wavelength of 280 nm.

TABLE 15
Primers used for the contruction of expression plasmids of the
LUZ19_gp5 truncation mutants, containing the phage gene with a N-
and/or C-terminal deletion, by site-directed mutagenesis.

A)

| | Truncation | Primer | |
|---|---|---|---|
| Vector | terminus | number | Primer sequence |
| pGEX-6P-1 | C-terminal | Primer 1 | TGAGAATTCCCGGGTCGACTCGAGCGGC |
| | | | [SEQ ID NO: 50] |
| | N-terminal | Primer 2 | CATGGATCCCAGGGGCC |
| | | | [SEQ ID NO: 51] |
| pUC18-mini- | C-terminal | Primer 3 | TGAAAGGGTGGGCGCGCCGAC |
| Tn7T-Lac-GW | | | [SEQ ID NO: 52] |
| | N-terminal | Primer 4 | CATGGTGAAGGGCTCCTTCTTAAAGTTAAAC |
| | | | [SEQ ID NO: 53] |

Overview of the used backbone primers, corresponding to the used
expression vector and the terminus at which the mutant LUZ19_gp5
is truncated
B)

| Truncation | | Δ amino | Primer | |
|---|---|---|---|---|
| terminus | Name | acids | number | Primer sequence |
| C-terminal | LUZ19-gp5^{Q70} | 71-74 | Primer 1 | CTGTCGGATGAATCGCACGTTGCGGGC |
| | | | | [SEQ ID NO: 54] |
| | LUZ19_gp5^{I68} | 69-74 | Primer 2 | GATGAATCGCACGTTGCGGGCCTCG |
| | | | | [SEQ ID NO: 55] |
| | LUZ19_gp5^{N64} | 65-74 | Primer 3 | GTTGCGGGCCTCGGCCTCG |
| | | | | [SEQ ID NO: 56] |
| | LUZ19_gp5^{E52} | 53-74 | Primer 4 | CTCACAGCGCACGTGGTAGATCGTCAT |
| | | | | ACAGC |
| | | | | [SEQ ID NO: 57] |
| N-terminal | LUZ19_gp5^{R8} | 1-7 | Primer 5 | CGCATCGGCCACCGC |
| | | | | [SEQ ID NO: 58] |

Overview of the coding primers used to create a deletion of the
selected amino acids at the N- or C-terminus of LUZ19_Gp5.

The nuclease activity of the LUZ19_Gp5 truncation mutants was studied similar to the nuclease activity of the wild-type LUZ19_Gp5 (see example 5). If no untagged protein was available, the activity test was performed with the N-terminal GST-tagged protein. However, since these samples were less pure (only affinity purification and no size-exclusion purification), the pre-incubation step of 1 h was performed at 90° C., which cannot inactivate LUZ19_Gp5-GST as discovered earlier (see example 4). Each test was performed in triplicate. When nuclease activity was detected at concentrations <5 μM of truncation mutant protein, the truncation mutant was classified as ‘active’. The results showed that LUZ19_gp5^{Q70}, LUZ19_gp5^{I68}, LUZ19_gp5^{R8} and LUZ19_gp5^{R8-Q70} still had in vitro nuclease activity, while the other truncations mutants were inactive.

TABLE 16
Yields and biochemical nuclease activity of purified recombinant LUZ19_Gp5
truncation mutant proteins. Yields are presented as milligrams protein per liter
E. coli expression culture as determined by spectrophotometric measurement
at 280 nm. Biochemical nuclease activity was assessed in a 20 μl reaction volume
by incubating increasing concentrations of the truncation mutant proteins with 100
ng λ DNA in a buffer mixture containing DNase I buffer + MgCl₂ for
1 h at 37° C. In case of untagged proteins, the incubation step was preceded
with a pre-incubation step without the DNA for 1 h at 37° C. Alternatively,
GST- tagged proteins were pre-incubated at 90° C. for 1 h without the
DNA. Truncation mutant proteins that displayed DNA-degradation at concentrations
below 5 μM protein were considered to have nuclease activity (✓).

| | | Expression yield | Nuclease |
|---|---|---|---|
| | Expression yield | untagged protein, | activity |
| Truncation | GST-tagged protein | after cleavage | detected 5 μM |
| mutant | [mg/l] | [mg/l] | protein |
| LUZ19_gp5^{Q70} | 18.21 | 0.30 | ✓ |
| LUZ19_gp5^{I68} | 1.41 | 0.009 | ✓ |
| LUZ19_gp5^{N64} | 0.17 | / | X |
| LUZ19_gp5^{E52} | 6.47 | 0.51 | X |
| LUZ19_gp5^{R8} | 13.73 | 0.52 | ✓ |
| LUZ19_gp5^{R8-Q70} | 0.58 | / | ✓ |

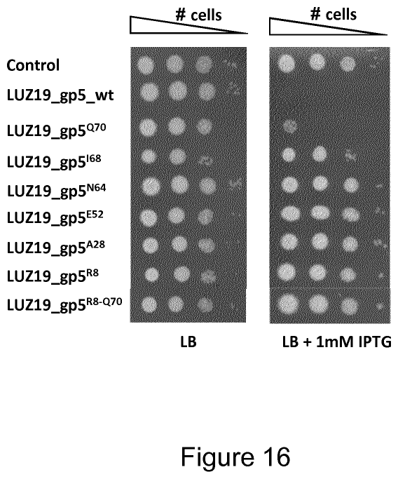

To evaluate the impact of the truncations on the in vivo toxicity of the nuclease in P. aeruginosa, the eight LUZ19_gp5 truncation mutants were expressed in P. aeruginosa PAO1. First, the pUC18-mini-Tn7T-Lac-GW plasmids were constructed by site-directed mutagenesis similar to the above described construction of the pGEX-6P-1 expression plasmids with truncated phage gene. For the site-directed mutagenesis, the same coding primers (table 15B: primers 1-6), but different backbone primers (table 15A, primers 3-4) were used and the pUC18-mini-Tn7T-Lac-GW vector containing the wild-type LUZ19_gp5 gene served as the template (see example 6). Single copy chromosomal integration of the truncated phage genes, as well as the assessment of the in vivo toxicity in P. aeruginosa PAO1 were performed exactly as described in example 6. The results showed that LUZ19_gp5^{Q70} was still toxic to P. aeruginosa, while all other truncation mutants did not inhibit the growth (FIG. 16).

FIG. 15 shows the selection of the LUZ19_gp5 truncation mutants. Based on the amino acid conservation of the homologs of LUZ19_gp5 and on the predicted secondary structure, seven truncation mutants were selected; five with a C-terminal deletion (LUZ19_gp5^{Q70}, LUZ19_gp5^{I68}, LUZ19_gp5^{N64}, LUZ19_gp5^{E52}, LUZ19_gp5^{A28}), and one with an N-terminal deletion (LUZ19_gp5^{R8}) and one with a deletion at both termini (LUZ19_gp5^{R8-Q70}) . Alignment of the homologs was visualized with ‘AlignmentViewer’ and secondary structures were predicted with ‘Sable secondary prediction server’.

FIG. 16 shows phenotypic effects of LUZ19_gp5 truncation mutant expression on P. aeruginosa PAO1 growth. A serial dilution of P. aeruginosa PAO1 cells, containing single-copy integration of the LUZ19_gp5 truncation mutant gene under control of an IPTG-inducible promotor, was spotted on solid LB media with (right) and without (left) 1 mM IPTG. As a negative control, a P. aeruginosa PAO1 strain, encoding an empty pUC18-mini-Tn7T-Lac expression cassette, was used.

### Example 14: Recombinant Expression of P. aeruginosa Phage Protein LUZ19_Gp5 in Pseudomonas putida. Cloning and Expression in P. putida KT2440

P. putida is a metabolic versatile bacterium that is increasingly used as a microbial cell factory to produce various relevant products (e.g. polyhydroxyalkanoates). Engineering of production strains can be used to recover the products in a more cost-effective and efficient way. For example, expression of nucleases can be used to degrade genomic DNA and thereby reduce viscosity in the product stream. In this regard, the activity of LUZ19_gp5 in P. putida KT2440 has been assessed by intracellular expression.

The phenotypical effect of expression of LUZ19_gp5, one toxic truncation mutant (LUZ19_gp5^{Q70}) and one non-toxic truncation mutant (LUZ19_gp5^{R8}) (see example 13) on the growth of P. putida KT2440 was evaluated. Co-transformation of 300 ng of the pUC18-mini-Tn7T-Lac-GW constructs, constructed as in example 6 and 13, and 500 ng pTNS2 plasmid by electroporation to P. putida KT2440 allowed single-copy integration of the phage gene and the truncation mutants in the P. putida genome under the control of an IPTG-inducible lac promotor. Protein expression to evaluate the phenotypical effect on the bacterial growth was performed similar to the evaluation in P. aeruginosa (see example 6 and 13). As a negative control, a P. putida KT2440 strain, encoding an empty pUC18-mini-Tn7T-Lac-GW expression cassette, was used. Results indicated that LUZ19_gp5 and its truncation mutants display the same phenotypical effect on the growth of P. putida and P. aeruginosa, namely LUZ19_gp5 and LUZ19_gp5^{Q70} cause growth inhibition upon intracellular expression, while LUZ19_gp5^{R8} does not inhibit bacterial growth.

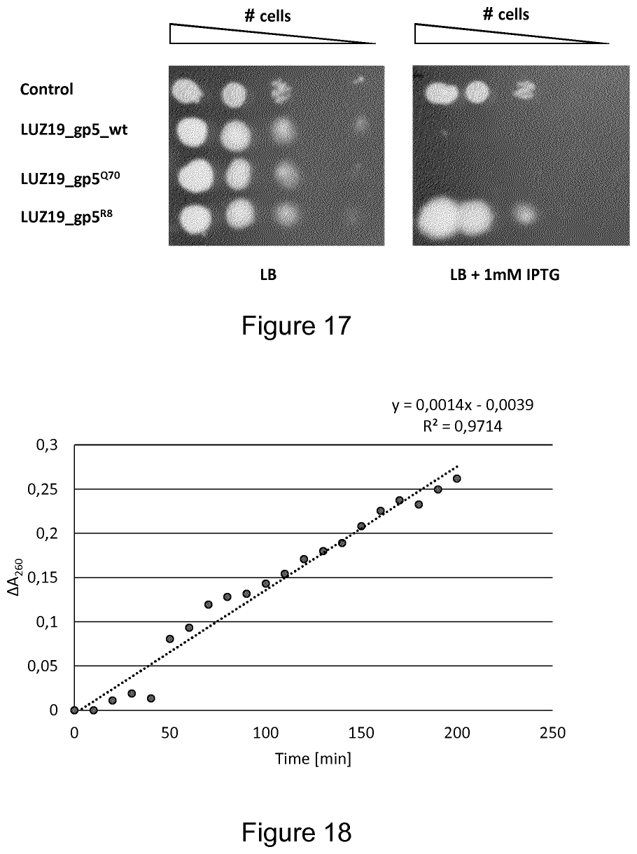

FIG. 17 shows phenotypic effects of the expression of LUZ19_gp5 and two truncation mutants on P. putida KT2440 growth. A serial dilution of P. aeruginosa PAO1 cells, containing single-copy integration of LUZ19_gp5 or the truncation mutant gene under control of an IPTG-inducible promotor, was spotted on solid LB media with (right) and without (left) 1 mM IPTG. As a negative control, a P. putida KT2440 strain, encoding an empty pUC18-mini-Tn7T-Lac expression cassette, was used.

### Example 15: Quantitative Assessment of the Biochemical Nuclease Activity of LUZ19_Gp5 by Spectrometry

The degradation of dsDNA by nucleases causes an increase in UV absorption at 260 nm (ΔA₂₆₀), referred to as hyperchromicity. This absorption increase allows to follow the degradation of dsDNA by nucleases in time and can therefore be used to quantitatively demonstrate the nuclease activity of LUZ19_Gp5. First, 38 μM of LUZ19_Gp5 was pre-incubated in DNase I buffer+MgCl₂ for 30 min at room temperature. Next, 50 μg/ml λ DNA was added, resulting in a final volume of 400 μl, and ΔA₂₆₀ was immediately assessed in a cuvette (UVette® 220 nm-1,600 nm, Eppendorf) using a spectrophotometer (BioPhotometer 6131, Eppendorf). A mixture containing only DNase I buffer+MgCl₂ and λ DNA was used as blank and measurements were taken every 10 minutes. A clear linear increase in absorption at 260 nm was observed, confirming the biochemical nuclease activity of LUZ19_gp5 in vitro (FIG. 18).

FIG. 18 shows the quantitative assessment of the in vitro nuclease activity of LUZ19_Gp5. The increase in UV absorption at 260 nm (ΔA₂₆₀) of a 400 μl mixture of 38 μM LUZ19_gp5 and 50 μg/ml λ DNA in DNase I buffer+MgCl₂ (pre-incubation without DNA; 30 min. T_{R}) at room temperature.

### Sequences Depicted in the Application

LUZ19_gp5
[SEQ ID NO: 1]
MSSRDPYRIG HRVGLVNYSD RYLGADAAGT KGTIEAITRP SRCMTIYHVR CERTLRLIEA
EARNVRFIRQ RAER
MPK6_gp5
[SEQ ID NO: 2]
MSSRDPYRIG HRVGLVNYSD RYLGADAAGT KGIIEAITRP SRCMTVYHVR CERTLRLIEA
EARNVRFIRQ RAER
vB_PaeP_PA01-Ab05_gp6
[SEQ ID NO: 3]
MSSRDPYRIG HRVGLVNYSD RYLGADAAGT KGIIEAITRP SRCMTIYHVR CERTLRLIEA
EARNVRFIRQ LPTRK
vB_Pae_TbilisiM32_gp5
[SEQ ID NO: 4]
MSSRDPYRIG HRVGLVNYSD RYLGADAAGT KGIIEAITRP SRCMTIYHVR CERTLRLIEA
EARNVRFIRQ LPTHK
DL62_gp8
[SEQ ID NO: 5]
MSSRDPYRIG HRVGLVNYSD RYLGADAAGT KGTIEAITRP SRCMTIYHVR CERTLRLIAA
EARNVRFVRQ LPTRK
MPK7_gp7
[SEQ ID NO: 6]
MSSRDPYRIG HRVGLVNYSD RYLGADAAGT KGIIEAITRP SRCMTVYHVR CERTLRLIEA
EARNVRFIRQ LPTRK
phikF77_gp6
[SEQ ID NO: 7]
MSSRDPYRIG HRVGLVNYSD RYLGADAAGT RGIIEAITRP SRCMTIYHVR CERTLRLIEA
EARNVRFIRQ LPTRK
phiKMV_gp5
[SEQ ID NO: 8]
MYSHDQYRIG HRVGLVNYSD RYLGADEAGT RGIVEAVTKP SRCMTVYHVR CEQTLRLIEA
EARNMRFIMQ PPTRK
LKD16_gp5
[SEQ ID NO: 9]
MFLHDRFDRH LGARVGLVNY SDRYLGADSA GVKGRIEAIT KPSRMMTVYH IRCEQTARLV
EAEEKNMRWL GR
vB_PaeP_PAO1_1-15pyo_gp6_full length
[SEQ ID NO: 10]
MKASTCCWSR AARCPATSPA RRTAGSTLNT TRAARTGARS STLQELRTMS SRDPYRIGHR
VGLVNYSDRY LGADAAGTKG IIEAITRPSR CMTIYHVRCE RTLRLIEAEA RNVRFIRQLP
THK
LUZ19_gp5
[SEQ ID NO: 11]
RIGHRVGLVN YSDRYLGADA AGTKGTIEAI TRPSRCMTIY HVRCERTLRL IEAEARNVRF I
MPK6_gp5
[SEQ ID NO: 12]
RIGHRVGLVN YSDRYLGADA AGTKGIIEAI TRPSRCMTVY HVRCERTLRL IEAEARNVRF I
vB_PaeP_PA01-Ab05_gp6
[SEQ ID NO: 13]
RIGHRVGLVN YSDRYLGADA AGTKGIIEAI TRPSRCMTIY HVRCERTLRL IEAEARNVRF I
vB_PaeP_TbilisoM32_gp5
[SEQ ID NO: 14]
RIGHRVGLVN YSDRYLGADA AGTKGIIEAI TRPSRCMTIY HVRCERTLRL IEAEARNVRF I
DL62_gp8
[SEQ ID NO: 15]
RIGHRVGLVN YSDRYLGADA AGTKGTIEAI TRPSRCMTIY HVRCERTLRL IAAEARNVRF V
MPK7_gp7
[SEQ ID NO: 16]
RIGHRVGLVN YSDRYLGADA AGTKGIIEAI TRPSRCMTVY HVRCERTLRL IEAEARNVRF I
phikF77_gp6
[SEQ ID NO: 17]
RIGHRVGLVN YSDRYLGADA AGTRGIIEAI TRPSRCMTIY HVRCERTLRL IEAEARNVRF I
PhiKMV_gp5
[SEQ ID NO: 18]
RIGHRVGLVN YSDRYLGADE AGTRGIVEAV TKPSRCMTVY HVRCEQTLRL IEAEARNMRF I
LKD16_gp5
[SEQ ID NO: 19]
HLGARVGLVN YSDRYLGADS AGVKGRIEAI TKPSRMMTVY HIRCEQTARL VEAEEKNMRW L
SEQ ID NO: 20
RVGLVNYSDRYLGAD
SEQ ID NO: 21
atgtcctcgc gtgatcccta ccgcatcggc caccgcgtgg ggctgatgaa ctacagcgac
cgctacctgg gtgccgacgc ggcaggcacc aagggcacca tcgaagccat aacccgaccg
tcccgctgta tgacgatcta ccacgtgcgc tgtgagcgga ccctgcgcct gatcgaggcc
gaggcccgca acgtgcgatt catccgacag cgggcggagc gg
SEQ ID NO: 22
atgtcctcgc gtgatccc
SEQ ID NO: 23
ccgctccgcc cgctgt
SEQ ID NO: 24
ataggatcca tgtcctcgcg tgatcc
SEQ ID NO: 25
atagaattct caccgctccg cccgctg
SEQ ID NO: 26
atgtcctcgc gtgatcccta ccgcatcggc caccgcgtgg ggctggtgaa ctacagcgac
cgctacctgg gtgccgacgc ggcaggcacc aagggcacca tcgaagccat aacccgaccg
tcccgctgta tgacgatcta ccacgtgcgc tgtgagcgga ccctgcgcct gatcgaggcc
gaggcccgca acgtgcgatt catccgacag cgggcggagc ggaagggtca tcatcaccat
caccattga
SEQ ID NO: 27
MSSRDPYRIG HRVGLVNYSD RYLGADAAGT KGTIEAITRP SRCMTIYHVR CERTLRLIEA
EARNVRFIRQ RAERKGHHHH HH
SEQ ID NO: 28
atgtccccta tactaggtta ttggaaaatt aagggccttg tgcaacccac tcgacttctt
ttggaatatc ttgaagaaaa atatgaagag catttgtatg agcgcgatga aggtgataaa
tggcgaaaca aaaagtttga attgggtttg gagtttccca atcttcctta ttatattgat
ggtgatgtta aattaacaca gtctatggcc atcatacgtt atatagctga caagcacaac
atgttgggtg gttgtccaaa agagcgtgca gagatttcaa tgcttgaagg agcggttttg
gatattagat acggtgtttc gagaattgca tatagtaaag actttgaaac tctcaaagtt
gattttctta gcaagctacc tgaaatgctg aaaatgttcg aagatcgttt atgtcataaa
acatatttaa atggtgatca tgtaacccat cctgacttca tgttgtatga cgctcttgat
gttgttttat acatggaccc aatgtgcctg gatgcgttcc caaaattagt ttgttttaaa
aaacgtattg aagctatccc acaaattgat aagtacttga aatccagcaa gtatatagca
tggcctttgc agggctggca agccacgttt ggtggtggcg accatcctcc aaaatcggat
ctggaagttc tgttccaggg gcccctggga tccccggaat tcccgggtcg actcgagcgg
ccgcatcgtg acctggaagt tctgttccag gggcccctgg gatccatgtc ctcgcgtgat
ccctaccgca tcggccaccg cgtggggctg gtgaactaca gcgaccgcta cctgggtgcc
gacgcggcag gcaccaaggg caccatcgaa gccataaccc gaccgtcccg ctgtatgacg
atctaccacg tgcgctgtga gcggaccctg cgcctgatcg aggccgaggc ccgcaacgtg
cgattcatcc gacagcgggc ggagcggtga
SEQ ID NO: 29
MSPILGYWKI KGLVQPTRLL LEYLEEKYEE HLYERDEGDK WRNKKFELGL EFPNLPYYID
GDVKLTQSMA IIRYIADKHN MLGGCPKERA EISMLEGAVL DIRYGVSRIA YSKDFETLKV
DFLSKLPEML KMFEDRLCHK TYLNGDHVTH PDFMLYDALD VVLYMDPMCL DAFPKLVCFK
KRIEAIPQID KYLKSSKYIA WPLQGWQATF GGGDHPPKSD LEVLFQGPLG SMSSRDPYRI
GHRVGLVNYS DRYLGADAAG TKGTIEAITR PSRCMTIYHV RCERTLRLIE AEARNVRFIR
QRAER
SEQ ID NO: 30
caccatgtcc tcgcgtgatc cc
SEQ ID NO: 31
tcaccgctccgcccgctg
SEQ ID NO: 32
atcatgccat accgcgaaag gttttgcacc a
SEQ ID NO: 33
ggaggggtgg aaatggagtt
SEQ ID NO: 34
gcaccatcgg agccataaccc
SEQ ID NO: 35
ccttggtgcc tgccgc
SEQ ID NO: 36
cggcaggccc caagggcac
SEQ ID NO: 37
cgtcggcacc caggtagcgg tcg
SEQ ID NO: 38
cataacccga ccgtcccgct g
SEQ ID NO: 39
gcttcgatgg ttctcttggt gcctgc
SEQ ID NO: 40
ataggatcca tgtactcgca tgacc
SEQ ID NO: 41
atagaattct catttacggg tcggc
SEQ ID NO: 42
ataggatcca tgttcttgca tgaccggttc
SEQ ID NO: 43
tatgaattct cagcgcccca gccag
SEQ ID NO: 44
MSSRDPYRIG HRVGLVNYSD RYLGADAAGT KGTIEAITRP SRCMTIYHVR CERTLRLIEA
EARNVRFIRQ
SEQ ID NO: 45
MSSRDPYRIG HRVGLVNYSD RYLGADAAGT KGTIEAITRP SRCMTIYHVR CERTLRLIEA
EARNVRFI
SEQ ID NO: 46
MSSRDPYRIG HRVGLVNYSD RYLGADAAGT KGTIEAITRP SRCMTIYHVR CERTLRLIEA
EARN
SEQ ID NO: 47
MSSRDPYRIG HRVGLVNYSD RYLGADAAGT KGTIEAITRP SRCMTIYHVR CE
SEQ ID NO: 48
MRIGHRVGLV NYSDRYLGAD AAGTKGTIEA ITRPSRCMTI YHVRCERTLR LIEAEARNVR
FIRQRAER
SEQ ID NO: 49
MRIGHRVGLV NYSDRYLGAD AAGTKGTIEA ITRPSRCMTI YHVRCERTLR LIEAEARNVR
FIRQ
SEQ ID NO: 50
tgagaattcc cgggtcgact cgagcggc
SEQ ID NO: 51
catggatccc aggggcc
SEQ ID NO: 52
tgaaagggtg ggcgcgccga c
SEQ ID NO: 53
catggtgaag ggctccttct taaagttaaa c
SEQ ID NO: 54
ctgtcggatg aatcgcacgt tgcgggc
SEQ ID NO: 55
gatgaatcgc acgttgcggg cctcg
SEQ ID NO: 56
Gttgcgggcc tcggcctcg
SEQ ID NO: 57
ctcacagcgc acgtggtaga tcgtcataca gc
SEQ ID NO: 58
cgcatcggcc accgc

1.-32. (canceled)

33. An in vitro method of degrading DNA, the method comprising: contacting a DNA comprising sample with a polypeptide comprising a sequence with at least 60% identity with any one of the sequences of SEQ ID NO: 1 to SEQ ID NO: 9 or a fragment thereof, wherein the polypeptide or fragment thereof has nuclease activity.

34. The method according to claim 33, wherein the method is performed at a pH below 4 or above 8.

35. The method according to claim 33, wherein the method is performed at a temperature above 70° C.

36. The method according to claim 33, wherein the polypeptide is a fusion protein.

37. The method according to claim 33, wherein the DNA comprising sample is a microbial sample.

38. The method according to claim 33, wherein the polypeptide comprises a sequence with at least 70% identity with any one of the sequences of SEQ ID NO: 1 to SEQ ID NO: 9 or a fragment thereof, and wherein the polypeptide or fragment thereof has nuclease activity.

39. The method according to claim 33, wherein the polypeptide comprises a sequence with at least 80% identity with any one of the sequences of SEQ ID NO: 1 to SEQ ID NO: 9 or a fragment thereof, and wherein the polypeptide or fragment thereof has nuclease activity.

40. The method according to claim 33, wherein the polypeptide comprises a sequence with at least 90% identity with any one of the sequences of SEQ ID NO: 1 to SEQ ID NO: 9 or a fragment thereof, and wherein the polypeptide or fragment thereof has nuclease activity.

41. A pharmaceutical composition comprising a polypeptide comprising a sequence with at least 60% identity with any one of the sequences of SEQ ID NO: 1 to SEQ ID NO: 9 or a fragment thereof, wherein the polypeptide or fragment thereof has nuclease activity.

42. A method of treating cystic fibrosis, the method comprising administering an effective amount of a polypeptide comprising a sequence with at least 60% identity with any one of the sequences of SEQ ID NO: 1 to SEQ ID NO: 9 or a fragment thereof, to an animal or human in need thereof, wherein the polypeptide or fragment thereof has nuclease activity.

43. The method according to claim 42, wherein the polypeptide comprises a sequence with at least 70% identity with any one of the sequences of SEQ ID NO: 1 to SEQ ID NO: 9 or a fragment thereof, and wherein the polypeptide or fragment thereof has nuclease activity.

44. The method according to claim 42, wherein the polypeptide comprises a sequence with at least 80% identity with any one of the sequences of SEQ ID NO: 1 to SEQ ID NO: 9 or a fragment thereof, and wherein the polypeptide or fragment thereof has nuclease activity.

45. The method according to claim 42, wherein the polypeptide comprises a sequence with at least 90% identity with any one of the sequences of SEQ ID NO: 1 to SEQ ID NO: 9 or a fragment thereof, and wherein the polypeptide or fragment thereof has nuclease activity.

46. A method of treating a bacterial infection, the method comprising administering an effective amount of a polypeptide comprising a sequence with at least 60% identity with any one of the sequences of SEQ ID NO: 1 to SEQ ID NO: 9 or a fragment thereof, to an animal or human in need thereof, wherein the polypeptide or fragment thereof has nuclease activity.

47. The method according to claim 46, wherein the polypeptide comprises a sequence with at least 70% identity with any one of the sequences of SEQ ID NO: 1 to SEQ ID NO: 9 or a fragment thereof, and wherein the polypeptide or fragment thereof has nuclease activity.

48. The method according to claim 46, wherein the polypeptide comprises a sequence with at least 80% identity with any one of the sequences of SEQ ID NO: 1 to SEQ ID NO: 9 or a fragment thereof, and wherein the polypeptide or fragment thereof has nuclease activity.

49. The method according to claim 46, wherein the polypeptide comprises a sequence with at least 90% identity with any one of the sequences of SEQ ID NO: 1 to SEQ ID NO: 9 or a fragment thereof, and wherein the polypeptide or fragment thereof has nuclease activity.

50. A polypeptide comprising a sequence with at least 60% identity with any one of the sequences of SEQ ID NO: 1 to SEQ ID NO: 9 or a fragment thereof, wherein the polypeptide or fragment thereof has nuclease activity, with the proviso that the polypeptide is not a polypeptide consisting of a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9.

51. The polypeptide according to claim 50, with the proviso that the sequence does not comprise a sequence consisting of any one of SEQ ID NO: 1 to SEQ ID NO: 9.

52. The polypeptide according to claim 50, with at least 70% identity with any one of the sequences of SEQ ID NO: 11 to SEQ ID NO: 19 or a fragment thereof, and wherein the polypeptide or fragment thereof has nuclease activity.

53. The polypeptide according to claim 50, with at least 80% identity with any one of the sequences of SEQ ID NO: 11 to SEQ ID NO: 19 or a fragment thereof, and wherein the polypeptide or fragment thereof has nuclease activity.

54. The polypeptide according to claim 50, with at least 90% identity with any one of the sequences of SEQ ID NO: 11 to SEQ ID NO: 19 or a fragment thereof, and wherein the polypeptide or fragment thereof has nuclease activity.

55. The polypeptide according to claim 50, which comprises the sequence RVGLVNYSDRYLGAD (SEQ ID NO: 20).

56. A polynucleotide comprising a sequence encoding a polypeptide according to claim 50.

57. An antibody specifically binding to a polypeptide comprising a sequence with at least 60% identity with any one of the sequences of SEQ ID NO: 1 to SEQ ID NO: 9 or a fragment thereof, wherein the polypeptide or fragment thereof has nuclease activity.

58. The antibody according to claim 57, wherein the antibody specifically binds to the sequence RVGLVNYSDRYLGAD (SEQ ID NO: 20).

59. A method of identifying modified versions of a polypeptide selected from the group consisting of SEQ NO: 1 to 9: with nuclease activity, the method comprising:
introducing one or more changes in the nucleotide sequence of a polynucleotide encoding a sequence selected from the group consisting of SEQ NO: 1 to 9, the one or more changes resulting in a modified amino acid sequence;
expressing the protein with the modified amino acid sequence; and
assaying the expressed protein for nuclease activity.
